(19) United States
(12) Patent Application Publication
(10) Pub. No.: US 2026/0260757 A1
(43) Pub. Date: Sep. 3, 2026

(54) POLYGENIC RISK ESTIMATOR FOR CERVICAL LENGTH CHANGE DURING PREGNANCY

(71) Applicant: VIRGINIA COMMONWEALTH UNIVERSITY, Richmond, VA (US)

(72) Inventors: Timothy YORK, Richmond, VA (US); Jerome STRAUSS, Richmond, VA (US)

(21) Appl. No.: 19/157,527

(22) PCT Filed: Feb. 28, 2024
(86) PCT No.: PCT/US2024/017631

§ 371 Date: Aug. 18, 2025

Related U.S. Application Data
(60) Provisional application No. 63/487,413, filed on Feb. 28, 2023.

(51) Int. Cl. G16H 50/30; C12Q 1/6809; C12Q 1/686; C12Q 1/6874; C12Q 1/6883; C12Q 1/689; G01N 33/68; G16B 20/20; G16H 10/60; G16H 20/10; G16H 50/70

(52) U.S. Cl. CPC G16H 50/30; C12Q 1/6809; C12Q 1/686; C12Q 1/6874; C12Q 1/6883; C12Q 1/689; G01N 33/689; G16B 20/20; G16H 10/60; G16H 20/10; G16H 50/70; C12Q 2600/106; G01N 2333/78

(57) ABSTRACT

A method for determining, for a woman, the risk of a premature short cervical length (sCL), and hence preterm birth, is provided. The method comprises measuring gene allelic variation in a biological sample obtained from the woman, developing a polygenic risk score (PRS), and identifying the woman as having an increased risk for sCL and preterm birth if the PRS is higher than that of corresponding standard controls. Methods for the prophylactic treatment of women identified as being at increased risk are also provided.

### CROSS-REFERENCE TO RELATED APPLICATIONS

This application claims benefit of United States Provisional Patent Application 63/487,413 filed Feb. 28, 2023.

### STATEMENT OF FEDERALLY SPONSORED RESEARCH AND DEVELOPMENT

This invention was made with government support under contract number HHSN275201300006C awarded by NICHD Perinatology Research Branch. The government has certain rights in the invention.

### BACKGROUND OF THE INVENTION

### Technical Field

The invention generally relates to methods of predicting which patients are at increased risk of aberrant cervical changes during pregnancy, in particular premature short cervical length (sCL) which can lead to spontaneous preterm birth. In particular, the invention provides a description of the genetic architecture of cervical length (CL) and a polygenic risk score (PGRS) to predict sCL during pregnancy, especially among women of African ancestry, who are at increased risk of preterm birth.

### Description of Related Art

The cervix is firm and closed during pregnancy which serves to protect the in utero environment from microorganism invasion and to support the developing fetus. Several weeks before parturition the cervix begins to efface, becoming softer and wider and decreasing in length. In most pregnancies delivered at term, a gradual decrease in cervical length (CL) begins at approximately 30 weeks gestational age. CL shortens from approximately 35-45 mm to complete effacement by the first stage of labor. In some women this process of cervical shortening begins earlier or happens more rapidly. A cervix shorter than 25 mm before 24 weeks of gestation is associated with a 6-fold increase in risk for a spontaneous preterm birth (i.e., birth before 37 completed weeks). The genetic and environmental mechanisms which control cervical dynamics across pregnancy are unclear. However, several maternal risk factors are associated with both a clinical short cervix (sCL) and preterm birth, including maternal age, parity, pre-pregnancy BMI, race (African ancestry, such as African American or Caribbean are especially at risk), and previous abortion.

There is a clear pathophysiological connection between cervical dysfunction and preterm birth. A recent study has shown that many maternal risk factors for preterm birth are mediated, at least in part, through their effects on cervical change during pregnancy. Whether genetic influences on preterm birth operate in a similar manner remains an open question. The estimated genetic contribution to preterm birth (11-35% fetal and 13-20% maternal heritability) may partially overlap with a genetic contribution to sCL and/or the rate of cervical effacement. If this is the case, then a woman's genetic profile, assessed even before pregnancy, could predict the risk of cervical dysfunction leading to preterm birth. This, by extension, could aid in identifying women with a high genetic burden for cervical dysfunction who could derive the greatest benefit from clinical intervention by pharmaceutical therapy—a strategy that has been proposed for other disease-drug combinations, including coronary heart disease and statin efficacy.

Sonographic cervical length (CL) in the mid-trimester of pregnancy is a robust predictor of spontaneous preterm birth (sPTB). While twin and family studies have established the contribution of genetic factors to preterm birth, there are no corresponding estimates of heritability for CL or an understanding of how genetic factors contribute to changes in the cervix across pregnancy. There is a pressing need for methods to determine the polygenic risk for cervical length change across pregnancy, especially in women of African ancestry.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Heretofore, there has been no characterization of the genetic architecture for CL or its rate of change during pregnancy. This is a particular need for women of African ancestry, such as African American or Caribbean women, who are at high risk of preterm birth. The present invention meets this need by providing tools and methods for determining a baseline risk for rapid cervical length change across pregnancy and its associated risk for short cervix leading to preterm birth. Exemplary aspects of the invention enable clinicians to predict which patients are at increased risk for aberrant cervical change across pregnancy, early cervix shortening and preterm birth using a polygenic risk score based on the genetic architecture of cervix length during pregnancy. A suite of genes or polymorphisms is identified and used to determine a polygenic risk score (PGRS). The level of risk for an individual woman is determined by comparison of her PGRS to the polygenic scores of corresponding control groups. The PGRS provides a baseline genetic risk assessment and, in some aspects, enhances existing clinical predictors of short cervix and preterm birth that do not consider a genetic contribution, enabling clinicians to predict which patients are at increased risk for aberrant cervical change across pregnancy, short cervix and preterm birth.

It is an object of this invention to provide a method of identifying and treating a woman for potential risk of preterm birth associated with mid-trimester clinical short cervix, comprising the steps of detecting in a biological sample from the woman prior to the women being in a mid-trimester of pregnancy a plurality of single nucleotide polymorphisms; calculating a polygenic risk score (PGRS) for the woman based on the detected plurality of single nucleotide polymorphisms; and treating, based on the calculated PGRS, the woman prior to the mid-trimester of pregnancy for the woman with one or more of a surgical procedure of cervical cerclage, or vaginally delivered progesterone. One or more additional risk features may be incorporated into the PGRS, including ancestry, ethnicity, age, weight, height, body mass index (BMI), previous live births, previous spontaneous preterm births (sPTB), abortions, miscarriages and a diagnosis of diabetes. Additional biomarkers may be incorporated including measures of fetal fibronectin, cervical elastography measurements, DNA methylation profiles of DNA extracted from blood and vaginal and cervical swabs, and microbiome abundance measures obtained from vaginal swabs. In one embodiment, the invention is a method of determining a PGRS for a woman of African ancestry. In another embodiment, the invention is a method of determining a PGRS for woman who is primigravida, or a woman is not pregnant during the detecting and calculating steps. In yet another embodiment, the invention is a method of determining a PGRS as described above, wherein the calculating step comprises extracting, into a diagnostic model, genetic features from a biological sample of the subject and mathematically integrating the genetic features into the diagnostic model to output the PGRS.

In one embodiment, the step of detecting is performed using whole genome sequencing. In another embodiment, detecting is performed using a microarray specific for said plurality of single nucleotide polymorphisms. In yet another embodiment, detecting is performed using polymerase chain reaction amplification of a suite of genes or genetic markers. The calculating step may be performed using polymorphism specific weights for different single nucleotide polymorphisms.

A variety of sample types may be used in practicing the method of the invention. Any biological comprising genetic material (e.g., genomic DNA, genes or gene products) is suitable for measurement. Accordingly, the biological sample is blood, plasma, buffy coat (i.e., white blood cells harvested from a blood sample), saliva, cervical cells, saliva, a vaginal sample, or any other bodily fluid or cells obtained from a subject in need of a PGRS.

In one embodiment, the method of the invention further comprises detecting a level of Saccharibacteria TM7-H1 in the vaginal sample and comparing of the detected level of Saccharibacteria TM7-H1 to a standard control. In this embodiment, treating is based on both the calculated PGRS and the comparison of the detected Saccharibacteria TM7-H1 to the standard control. The level of Saccharibacteria TM7-H1 in a vaginal sample obtained from the woman is compared to the detected level of Saccharibacteria TM7-H1 in a standard control, and treating is based on both the calculated PGRS and the comparison of the detected Saccharibacteria TM7-H1 to the standard control.

In another embodiment, the method of the invention further comprises a fetal fibronectin assay. The clinical utility of the fetal fibronectin test relates to its high negative predictive value to identify women who are at low risk of preterm delivery. Thus, a combination of an assay of a suite of genes or polymorphisms and a fetal fibronectin assay has increased predictive power to identify a woman's risk of preterm delivery.

In another embodiment, the invention is a method of determining a risk of mid-trimester clinical short cervix (sCL) for a woman in need thereof and treating the woman, comprising identifying the woman as being at risk for preterm birth by the steps of detecting in a biological sample from the subject the presence or absence of alleles of common allelic variants associated with preterm birth, wherein the step of detecting is performed during a first trimester of pregnancy; calculating a polygenic risk score (PGRS) for said subject based on the presence or absence of the alleles of common allelic variants; identifying the subject as being at risk for preterm birth when the PGRS score differs from at least one control PGRS score; and treating the woman identified as being at risk for preterm birth by one or more of administering progesterone, bed rest, cervical cerclage, or cervical pessary.

In yet another embodiment, the invention is a method for determining a risk of preterm birth for a woman in need thereof and treating the woman, comprising identifying the subject as being at risk for preterm birth by detecting in a biological sample from the subject the presence or absence of alleles of common allelic variants associated with preterm birth, wherein the step of detecting is performed during a first trimester of pregnancy; calculating a polygenic risk score (PGRS) for said subject based on the presence or absence of the alleles of common allelic variants; identifying the subject as being at risk for preterm birth when the PGRS score differs from at least one control PGRS score; and treating the subject identified as being at risk for preterm birth by one or more of administering progesterone, bed rest, cervical cerclage, or cervical pessary.

### BRIEF DESCRIPTION OF THE DRAWINGS

FIG. 1. Serial measurements of CL within individuals across pregnancy. Marginal plots show distribution of CL data across gestational ages and birth outcome classes. Average trajectories of CL change during pregnancy estimated by LGCA are shown for four classes of clinical birth outcomes (extremely preterm births delivered before 28 weeks gestation, very preterm births delivered before 32 weeks gestation, late preterm births delivered before 37 weeks gestation, and full-term births delivered after 37 completed weeks of gestation) are shown in gray shading. Note the difference in the shape and rate of cervical change during pregnancy varies by gestational age at delivery.

FIG. 2A-C. Heritability estimates for: A. birth outcome and biometric traits within the study cohort B. heritability estimates for birth outcome and biometric traits in the study cohort compared to heritability estimates of the same traits in the published literature, and C. Genetic correlations between cervical length growth parameters (I, L, and Q) and gestational outcomes (GAD and sPTB). Note that negative genetic correlations occur when an increase in trait 1 is correlated with a decrease in trait 2.

FIGS. 3A and B. A, ROC Curves for prediction of sCL in the full testing sample (N=827) using clinical, genetic, and combined clinical+genetic predictors. Clinical predictors included maternal age, pre-pregnancy BMI, parity, number of previous preterm births, and number of previous abortions. Prediction of sCL in primiparous women used maternal age and pre-pregnancy BMI as clinical predictors; B. ROC Curves for prediction of sCL in primigravida in the testing sample (N=301) using clinical, genetic, and combined clinical+genetic predictors; C. clinical, genetic, and combined clinical+genetic predictors for predicting sCL in primigravada (N=301) vs. multigravida (N=526) pregnancies; D. Values for the AUC, sensitivity, specificity, and actual versus predicted outcomes for sCL using the full testing sample (N=827) as well as the primigravida subgroup (N=301) and multigravida subgroup (N=526).

FIG. 4A-D. Manhattan plot (N=4456) showing the location and genome-wide association results for SNPs where the red line delineates the genome-wide significance threshold, and the blue line delineates suggestive significance threshold for, A CL intercept term (I) B CL linear term (L), C CL quadratic term (Q), and D gestational duration/sPTB.

FIG. 5. Principal Component Analysis (PCA) scatter plot showing the distribution of individuals based on the first two principal components (PC1 and PC2). Each point represents an individual, with colors indicating their self-reported race. The plot illustrates the genetic diversity within and between racial groups and highlights the genetic structure captured by PC1 and PC2.

FIG. 6. Enriched differentially expressed gene (DEG) sets from GTEx v8 54 tissue types.

### DETAILED DESCRIPTION

A unique data set of serial CL measurements in a sample of black women was used to answer fundamental questions regarding the role that genetic variation contributes to inter-individual differences in cervical dysfunction. The studies described herein successfully characterized the genetic architecture for CL and its rate of change, thereby providing, first, an estimate of heritability of CL change across pregnancy, and second, the ability to predict the risk for a sCL by developing a polygenic risk score based on genetic variants associated with CL change during pregnancy. Using the methods and tools disclosed herein, clinicians can predict which patients are at increased risk for aberrant cervical change across pregnancy, in particular, early (premature) shortened cervix and preterm birth. This polygenic risk score provides both a baseline genetic risk assessment and an enhancement of existing clinical predictors of short cervix and preterm birth that have not heretofore been considered a genetic contribution.

In one embodiment, genotype data from patient DNA specimens subjected to NGS 1× low-pass whole genome sequencing or microarray is required to assess genotypes for a plurality of single nucleotide polymorphism values. DNA specimens can be extracted from either easily accessible saliva or blood samples, the latter being commonly obtained at prenatal care visits. Saliva samples could be obtained from at-home collection kits and mailed in to a diagnostic lab for assessment of the risk profile. Polygenic scores (PGS) for patient genotypes are computed by the predictor algorithm based on polymorphism specific weights which yields a risk probability for developing a short cervix. Pregnant women classified as high-risk for developing a short cervix would initially be referred to a Maternal Fetal Medicine (MFM) specialist and most likely will be accompanied by an increase in cervical length monitoring. The MFM specialist would have additional care options including the administration of vaginal progesterone and/or cerclage to maintain the appropriate length and integrity of the cervix throughout the remaining gestational period.

### Definitions

A genetic signature is information about the activity of a specific group of genes in a cell or tissue. Gene signatures can show how likely certain types of conditions or disease are to occur. A gene signature may be used to help diagnose diseases/conditions and plan treatment. Similarly, the present disclosure encompasses protein signatures and epigenetic signatures having the same function.

“Genetic architecture” is a term of art that refers to the underlying genetic basis of a phenotypic trait and its variational properties. Phenotypic variation for quantitative traits is, at the most basic level, the result of the segregation of alleles at quantitative trait loci (QTL). Genetic architecture can be described for an individual based on information regarding, for example, gene and allele number, the distribution of allelic and mutational effects, patterns of pleiotropy, dominance, and epistasis. Genetic architecture is studied and applied at many different levels. At the most basic, individual level, genetic architecture describes the genetic basis for differences between individuals, species, and populations.

A “locus” (plural “loci”) corresponds to an identified location in a genome and can span a single base or a sequential series of multiple bases. A locus is typically identified by using an identifier value or a range of identifier values with respect to a reference genome and/or a chromosome thereof. A “region” in a genome may include one or more loci. A “heterozygous locus” (also referred to as a “het”) is a locus in a genome, where the two copies of a chromosome do not have the same sequence. These different sequences at a locus are called “alleles”. A het can be a single-nucleotide polymorphism (SNP) if the reference genome location has two alleles that differ by a single base. A “het” can also be a reference genome location where there is an insertion or a deletion (collectively referred to as an “indel”) of one or more nucleotides or one or more tandem repeats. A “homozygous locus” is a locus in a reference or a baseline genome, where the two copies of a chromosome have the same allele. “Haplotype” refers to a set of DNA variants along a single chromosome that tend to be “linked” or inherited together.

A polygenic score (PGS) is a number that summarizes the estimated effect of many genetic variants on an individual's phenotype. The PGS is also called the polygenic index (PGI) or genome-wide score; in the context of disease risk, it is called a polygenic risk score (PGRS or PR score or genetic risk score). The score reflects an individual's estimated genetic predisposition for a given trait and can be used as a predictor for that trait. It gives an estimate of how likely an individual is to have a given trait based only on genetic factors, without taking environmental factors into account; and it is typically calculated as a weighted sum of trait-associated alleles.

“Preterm birth” is defined as babies born alive before 37 weeks of pregnancy are completed. There are sub-categories of preterm birth, based on gestational age: extremely preterm (less than 28 weeks) very preterm (28 to less than 32 weeks) moderate to late preterm (32 to 37 weeks).

A short cervix is one that is less than 25 mm (2.5 cm) long at around 20 weeks of pregnancy. The shorter the cervix, the higher the risk of an early (premature) birth (earlier than 37 weeks pregnancy).

Single nucleotide polymorphisms (SNPs) are defined as loci with alleles that differ at a single base, with the rarer allele having a frequency of at least 1% in a random set of individuals in a population.

Structural variants” involve changes in some parts of the chromosomes instead of changes in the number of chromosomes or sets of chromosomes in the genome. There are four common types of mutations which result in structural variants: deletions and insertions, for example duplications (involving a change in the amount of DNA in a chromosome, loss and gain of genetic material, respectively), inversions (involving a change in the arrangement of a chromosomal segment) and translocations (involving a change in the location of a chromosomal segment which can give rise to gene fusions). In the present invention, the term “structural variant” includes loss of genetic material, a gain of genetic material, a translocation, a gene fusion and combinations thereof.

As used herein, the term “variation” refers to a change or deviation. In reference to nucleic acid, a variation refers to a difference(s) or a change(s) between DNA nucleotide sequences, including differences in copy number (CNVs). This actual difference in nucleotides between DNA sequences may be an SNP, and/or a change in a DNA sequence, e.g., fusion, deletion, addition, repeats, etc., observed when a sequence is compared to a reference, such as, e.g., germline DNA (gDNA) or a reference human genome HG38 sequence. Information on short genetic variations can be obtained using NCBI's SNP database (dbSNP) using Ref SNP (rs) numbers. Information on large structural variations, e.g., insertions, deletions, duplications, inversions, mobile elements, and translocations can be obtained using NCBI's variation database (dbVar) using an NCBI (nsv) or EBI (esv) reference number.

Additional definitions pertinent to genetic analyses and the development of PGS are found, for example, in published United States patent applications US20130224117, US20210071255 and US20200027557, the complete contents of each of which is hereby incorporated by reference in entirety.

### Developing Standard Gene Signatures for sCL

The genetic information that is used to develop an initial or standard controls on which individual PGRSs are eventually based is typically obtained, for example, by detecting the nucleic acid sequence of one or more (at least one) nucleotide sequence of interest at loci of interest. Nucleotide sequences of interest may be genes, segments of genes, SNPs (which may be located within but are generally located outside a gene), RNA molecules, or segments of any of these, etc. Generally, a statistically significant plurality of sequences is obtained, and preferably whole human genome sequencing, or whole genome sequence databases, are employed to obtain sequence data. Those of skill in the art will recognize that it may or may not be necessary to sequence an entire gene, especially once key features of a gene of interest are found, e.g., an associated mutation. The detection of short sequences which encompass key nucleotide sequences may suffice.

Numerous sequence databases are available for public use and searching. Alternatively, numerous commercial facilities also provide this service for a fee. Sequencing used for the present disclosure may include whole genome, whole exome, targeted sequencing, multiple displacement amplification, etc. Polymerase chain reaction (PCR) may also be used to amplify segments of genomic DNA and identify one or more variants or a suite of variants in a genome.

What is looked for (determined) in the plurality of sequences is patterns of genes and/or genetic mutations and/or alleles and/or variants and/or patterns of gene expression, that are indicative of (characteristic of, associated with, are biomarkers of, etc.) a particular trait. In the present case, the trait is undesirable premature cervical shortening during pregnancy. Indications of such associations are generally identified by comparing a statistically significant number of samples from subjects known to have the trait (a positive control) with a statistically significant number of samples from subjects known to not have the trait (negative control). Herein, a positive control group is typically a group of women, usually of African ancestry, who are already known to have had undesirable early cervical shortening during pregnancy and/or who have already experienced preterm birth of at least one child. Conversely, a negative control group is typically a group of women, usually of African ancestry, who have never had undesirable, early cervical shortening during pregnancy and/or who have delivered at least one child without experiencing preterm birth.

Those of skill in the art will recognize that developing categories of samples to serve as positive and negative controls may include other populations as well. This may include various racial and/or ethnic backgrounds. For example, the racial and/or ethnic backgrounds of the positive and negative controls may be matched with the racial and/or ethnic background of the patient. In addition, the categories may be various subsets of populations. For example, another negative control could be women who have never been pregnant.

Further, the control populations may be more nuanced such as, for example, a control population of women who have experienced early cervical shortening but not preterm birth, women who have had multiple children and sometimes have had preterm birth and sometimes have not, etc. The sequence variation data that is obtained may be analyzed using any parameters or filters that are useful for developing control gene signatures.

Generally, a minimum of at least about 5,000 loci are sequenced/detected, for example from about 100 to about 10,000 are sequenced, in order to establish initial control gene signatures (PGSs) that are associated with a trait of interest. In some aspects, at least about 100, 500, 1000, 1500, 2000, 2500, 3000, 3500, 4000 or about 4500 SNPs are used; and/or about 100, 200, 300, 400 or 500 SNPs; and/or about 100, 200, 300, 400, or 500 genomic risk loci; and/or about 100, 200, 300, 400, 500, 600, 700, 800, 900, 1000, 1100, 1200, 1300, 15400, 1500, 1600, 1700, 1800, 1900 or 2000 mapped genes are identified. The signature according to certain embodiments of the present invention may comprise or consist of one or more genes, gene sets, proteins, protein families, loci, and/or epigenetic elements, such as for instance at least one of those listed in Tables 4-13. In other aspects, the loci include rs75296056 and rs185443461 on chromosome 18 (p=1.85×10⁻⁹) from a region near the gene SETBP1 (SET binding protein 1), which is highly expressed in cervical tissue, and interacts with the estradiol pathway.

Methods of identifying genetic variants is well known in the art and may be performed using a variety of techniques, many of which are provided by sequencing companies or facilities that employ software designed to analyze large collections of genetic data, e.g. databased of the human genome. Thus, once candidate sequences are identified, they are processed by an analysis program capable of assessing large amounts of data in a statistically significant manner. Modeling and analysis is performed by any suitable method, examples of which include but are not limited to genetic association tests and/or latent growth curve modeling/analyses, and any others that can be used to characterize the change in cervical length across pregnancy.

In some aspects, the analysis is performed using latent growth curve modeling. Latent growth curve modeling is known in the art and is described, for example, in published United States patent applications US20130224117, US20210071255 and US20200027557, the complete contents of each of which is hereby incorporated by reference in entirety.

Other factors may be used to develop the controls or standards, including but not limited to: maternal age, weight, height, parity, pre-pregnancy body mass index (BMI), previous pregnancy outcomes (previous spontaneous preterm births (sPTB), or previous miscarriages/terminations or previous live births), and diagnoses of diabetes. Additional biomarkers could be incorporated including measures of fetal fibronectin, cervical elastography measurements, DNA methylation profiles of DNA extracted from blood and vaginal and cervical swabs, and microbiome abundance measures obtained from vaginal swabs.

In addition, the method may optionally include a fetal fibronectin assay. The clinical utility of the fetal fibronectin test relates to its high negative predictive value to identify women who are at low risk of preterm delivery. Thus, a combination of an assay of a suite of genes or polymorphisms and a fetal fibronectin assay has increased predictive power to identify a woman's risk of preterm delivery. Fetal fibronectin tests known in the art and are described in, for example, Ikeoha et al. (BioMed research International. Vol. 2022, Article ID 2442338, 9 pages). See also Berghella and Saccone (Cochrane Database of Systematic Reviews. 2019, Issue 7, Art. No.: CD006843). Fetal fibronectin is an extracellular matrix glycoprotein. Fetal fibronectin in biologic fluids is produced by amniocytes and by cytotrophoblast. It is present throughout gestation in all pregnancies. In general, fetal fibronectin is found in vaginal fluids early in pregnancy because of the normal growth and establishment of tissues at the junction between the amniotic sac and uterus, with levels falling when this phase is complete. It is not subject to genetic polymorphism. There are very high levels in amniotic fluid (100 Sg/mL) in the second trimester, and 30 Sg/mL at term. It is localized at the maternal-fetal interface of the amniotic membranes, between chorion and decidua, where it is concentrated in this area between decidua and trophoblast. Here it acts as a ‘glue’ between the pregnancy and the uterus. The concentration of fetal fibronectin protein found in blood is ⅕th that found in amniotic fluid; it is not present in urine. In normal conditions, this glycoprotein remains in this area between chorion and decidua, and very low levels are found in cervicovaginal secretions after 22 weeks (less than 50 ng/mL). Levels above this value (greater than or equal to 50 ng/ml) at or after 22 weeks in the cervicovaginal secretions collected by a swab have been associated with an increased risk of spontaneous preterm birth. The test for fetal fibronectin is typically performed as an immunoassay, such as an enzyme immunoassay, antibody-based immunoassay or solid-phase immunogold assay. Commercially available tests, such as Rapid fFN® for the TLiiq® System (Hologic, Inc.; Marlborough MA) are suitable.

As an alternative, in some aspects, what is detected (measured, identified, etc.) is the amino acid sequence of one or more proteins, protein fragments, polypeptides or peptides that are encoded by and expressed in full or in part by one or more genes of interest. In some aspects, the amino acid sequences correspond to genetic sequences identified as described above.

### Assigning an Individual PGRS to a Woman

Once control or standard PGSs or ranges thereof have been established, an individual woman's risk of experiencing an early cervix shortening during pregnancy can be determined by comparison thereto.

In some aspects, the woman who is tested using the present methods is a woman of African ancestry who is pregnant. In other aspects, the woman who is tested using the present methods is a woman of African ancestry who is not yet pregnant but who could benefit from the information provided by the test for future reference and planning. In some aspects, the woman is a primigravida woman who is pregnant with her first child. In other aspects, the woman is a multigravida woman who has previously been pregnant with at least one other child. In particular, the disclosed methods are helpful for identifying primigravida women who do not have an obstetric history, in order to inform them and their health care providers of their risk of developing a sCL and/or delivering preterm, and providing treatment. This is especially important because primigravida women are generally less likely to receive mid-trimester CL screening.

The sample from a woman that is utilized is any biological sample from which suitable genetic information can be obtained, i.e. from which the selected signature genes characteristic of sCL and be detected. Exemplary samples include but are not limited to: blood, plasma, buffy coat samples (i.e., white blood cells harvested from a blood sample), cervical cells, vaginal fluid and saliva.

Once the genetic information (e.g. the presence of alleles, mutations, etc. that are characteristic of sCL) is obtained from a subject, the result for the subject is compared to the control gene signatures that have been established for sCL.

The result of the comparison is typically reported as a polygenic risk score (PGRS) that indicates the statistical likelihood (risk) that the woman will experience sCL during pregnancy. If the likelihood (risk) of sCL is high, then the woman is also likely to experience preterm birth. Conversely, if the likelihood (risk) of sCL is low, then the woman is not likely to experience preterm birth.

The complete contents of U.S. application Ser. No. 17/608,242 are hereby incorporated by reference in its entirety. U.S. application Ser. No. 17/608,242 discloses methods for determining the risk of an adverse pregnancy outcome, e.g., a preterm birth, comprising the steps of measuring a level of Saccharibacteria TM7-H1 and optionally one or more of BVAB1, Sneathia amnii, and Prevotella cluster 2, in a vaginal sample obtained from a woman and identifying the woman as having an increased risk for an adverse pregnancy outcome if the levels are increased compared to corresponding standard control levels. Thus, in one embodiment, the method of the invention further comprises detecting a level of Saccharibacteria TM7-H1 in the vaginal sample and comparing of the detected level of Saccharibacteria TM7-H1 to a standard control. In this embodiment, treating is based on both the calculated PGRS and the comparison of the detected Saccharibacteria TM7-H1 to the standard control. The level of Saccharibacteria TM7-H1 in a vaginal sample obtained from the woman is compared to the detected level of Saccharibacteria TM7-H1 in a standard control, and treating is based on both the calculated PGRS and the comparison of the detected Saccharibacteria TM7-H1 to the standard control. Thus, the final PGRS obtained for the subject is a combination of genetic analysis and TM7-H1 levels.

Those of skill in the art will recognize that “high” and “low” are relative terms and are generally developed in relation to predetermined, corresponding gene signatures using statistical analysis techniques. A PGRS is typically a single number that falls on a standard or control scale or range of probability. For example, the control scale may assign women who have been found to be at the highest risk (based on their genetic profiles, as described above) as a “10” and those who have the lowest risk genetic profile as a “1”. The scale may indicate (be interpreted as) that a woman having a PGRS greater than 5 is at high risk, or that a woman having a PGRS of from 5-7 is at moderate risk and a woman having a PGRD of 8-10 is at high risk, etc. Alternatively, the comparison may yield a percentage risk for the woman, e.g. the woman with a PGRS of 5 may have a 50% risk of sCL while a PGSR of 8 indicates an 80% chance. The percentage risk may be considered as having a level of increased risk assigned to 40%, 50%, 60%, 70%, 80%, 90% or more, and any of these may be designated as a threshold representing an increased risk when the score exceeds the threshold. It is to be understood that these numbers are exemplary only and that scales of e.g., 1-100, or 1-1000 may be used, and many categories may be established (e.g. low, moderately low, moderately high, high, etc.) may be established.

Further, as described above, other factors may be employed to make the score more accurate. For example, a history of previous preterm births would likely increase the risk of another preterm birth. Such additional information can be included before a PGRS is determined (i.e., built into the PGRS) or may be taken into account after a PGRS is determined, to result in, for example a composite score (the PGRS score based on genetics plus one or more other risk factors).

### Treatments

There are several treatment options available for a woman who is identified as having a high likelihood of premature cervical shortening and thus premature birth. Treatment options or ways to treat and/or manage a short, incompetent cervix include but are not limited to:
Bed rest and/or frequent monitoring. Bed rest can delay cervical effacement. Frequent monitoring, such as frequent imaging (e.g., weekly, biweekly) of the cervix can be performed. Repeated sonograms (ultrasounds) may be used to monitor the cervical length and the status of the fetus.
Progesterone supplementation. If the woman has a short cervix with no history of a preterm birth, vaginal progesterone administration may lower the risk of preterm birth.
Cervical cerclage. involves temporarily sewing the cervix closed with stitches to help the cervix hold a pregnancy in the uterus. A cerclage is typically done in the second trimester of pregnancy to prevent preterm birth.
Cervical pessary. The pessary is a device that fits around a cervix to keep it from opening and leading to miscarriage or preterm birth. The type and size of the pessary is fitted to each individual's needs and anatomy.

In view of the inconveniences and/or risks involved with each of these treatments, the method of the invention provides benefits to both a woman and a clinician by identifying conditions where there is an increased risk of cervical shortening and pre-term birth.

### OTHER ASPECTS

This disclosure also relates to systems, software and methods for diagnosis or prognosis of subjects for sCL and/or preterm birth, including, classification and treatment of subjects who have been diagnosed with or deemed at risk of sCL and/or preterm birth. The methods are based, in part, on the multimodal analysis of a plurality of features, e.g., genetic features such as SNPs or chromosome regions, including loci and/or genes related thereto and, optionally, other factors such as those disclosed elsewhere herein, e.g., BMI, past history of preterm births, etc.

Accordingly, in some aspects, the disclosure relates to a computer readable medium comprising computer-executable instructions, which, when executed by a processor, cause the processor to carry out a method or a set of steps for predicting a risk of sCL and/or preterm birth in a subject, the method or steps comprising, a) extracting, into a diagnostic model, a plurality of features comprising genetic features from the subject's biological sample and optionally other known risk factors (e.g., previous preterm births); b) mathematically integrating the genetic features in the diagnostic model to output a first integrated score; c) optionally integrating the other known risk factors in the diagnostic model to output a second integrated score and d) outputting a risk score (PGRS) based on the first, and/or second integrated scores.

In some embodiments, the genetic features comprise at least one or all of the genetic features of Tables 4-13, or any of various subsets thereof.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are described herein; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Polygenic Scores Predict Mid-Trimester Short Cervix

This study investigates the contribution of genetic variation to inter-individual differences in cervical change in a large US-based sample of women with serial CL measurements across pregnancy. Since there is no characterization of the genetic architecture for CL or its rate of change during pregnancy in the literature, this study focused on three primary goals. First, to provide the first estimate of heritability for CL change across pregnancy; second, to quantify the genetic relationship between cervical change and gestational duration and third, to predict risk for a sCL by developing a polygenic score based on genetic variants associated with CL change across pregnancy.

### Methods

### Cohort Description and Genotyping

The study cohort is composed of self-identified Black/African-ancestry women from the Detroit, Michigan area enrolled in a prospective cohort study of pregnant women at the Center for Advanced Obstetrical Care and Research (CAOCR) at Hutzel Women's Hospital. Clinical data collection was approved by the Institutional Review Boards of Wayne State University (#110605MP2F) and NICHD/NIH/DHHS (OH97-CH-N067). DNA specimens provided by 4,456 women were sent to BGI Group Next Generation Sequencing Services for 1× low-pass, whole genome sequencing, passed all quality control measures, and contained the relevant phenotypic data for inclusion in the genetic analyses. Approximately 16 million single-nucleotide polymorphisms (SNPs) remained after quality control (See Supplemental Methods below). Cohort demographic information by gestational duration status is presented in Table 1.

TABLE 1
Cohort Demographic Table

| | OVERALL | | PRETERM | | TERM | | |
|---|---|---|---|---|---|---|---|
| Variable | N = 4,456 | | N = 566 | | N = 3,890 | | P-VALUE² |
| Age | 23.0 | (20.0, 27.0) | 23.0 | (20.0, 28.0) | 23.0 | (20.0, 27.0) | 0.03 |
| Self-Reported | | | | | | | 0.8 |
| Race/Ethnicity | | | | | | | |
| Black | 4,137 | (93%) | 532 | (94%) | 3,605 | (93%) | |
| Other | 318 | (7%) | 34 | (6%) | 285 | (7%) | |
| Maternal | 64.0 | (62.0, 66.0) | 64.0 | (62.0, 66.0) | 64.0 | (62.0, 66.0) | 0.2 |
| Height (in) | | | | | | | |
| Pre-Pregnancy | 27 | (23, 33) | 27 | (22, 33) | 27 | (23, 33) | 0.4 |
| BMI | | | | | | | |
| Parity | | | | | | | >0.9 |
| 0 | 1,932 | (51%) | 234 | (50%) | 1,698 | (51%) | |
| 1 | 1,188 | (31%) | 142 | (31%) | 1,046 | (31%) | |
| 2+ | 698 | (18%) | 88 | (19%) | 610 | (18%) | |
| Unknown | 638 | | 102 | | 536 | | |
| Previous | | | | | | | <0.001 |
| Preterm | | | | | | | |
| Delivery | | | | | | | |
| 0 | 3,930 | (89%) | 407 | (75%) | 3,523 | (91%) | |
| 1 | 404 | (9.1%) | 105 | (19%) | 299 | (7.7%) | |
| 2+ | 83 | (1.9%) | 31 | (5.7%) | 52 | (1.3%) | |
| Unknown | 39 | | 23 | | 16 | | |
| Previous | | | | | | | 0.8 |
| Abortions | | | | | | | |
| 0 | 2,305 | (57%) | 279 | (56%) | 2,026 | (57%) | |
| 1 | 1,176 | (29%) | 151 | (31%) | 1,025 | (29%) | |
| 2+ | 537 | (13%) | 64 | (13%) | 473 | (13%) | |
| Unknown | 438 | | 72 | | 366 | | |
| GAD | 39.10 | (38.0, 40.10) | 35.0 | (32.30, 36.30) | 39.30 | (38.60, 40.30) | <0.001 |
| sCL | | | | | | | <0.001 |
| Normal CL | 3,528 | (95%) | 367 | (84%) | 3,161 | (97%) | |
| Short CL | 174 | (4.7%) | 68 | (16%) | 106 | (3.2%) | |

| | | | |
|---|---|---|---|
| Unknown | 754 | 131 | 623 |

### Assessment of Cervical Length Change Across Pregnancy

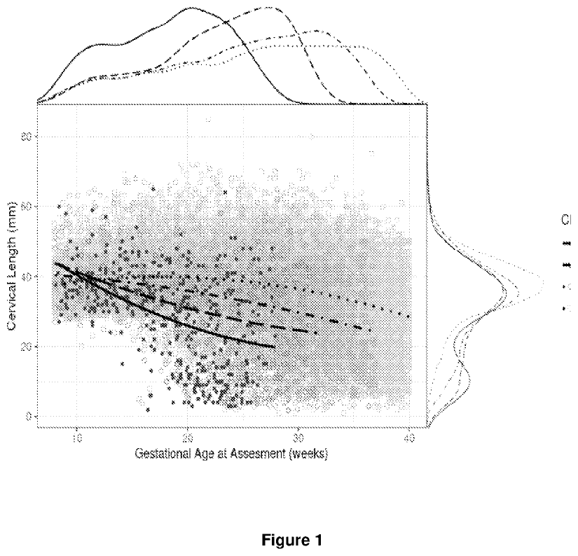

Serial sonographic CL measurements were available for all participants and were used to quantify linear and non-linear characteristics of cervical change during pregnancy using Latent Growth Curve Analysis (LGCA). LGCA is a cross-disciplinary analytic approach that leverages repeated measurements to calculate how a trait changes within and between individuals over time. Interindividual differences in trajectories of cervical change were described by intercept (I), linear (L), and quadratic (Q) growth curve parameters. The I, L, and Q parameters estimated for each pregnancy in the cohort were used as phenotypes for all genetic analyses. Details of this LGCA analysis have previously been described (FIG. 1; Wolf et al. BMJ Open 2022; 12 (3): e053631).

### Trait Heritability and Bivariate Genetic Correlation Estimation

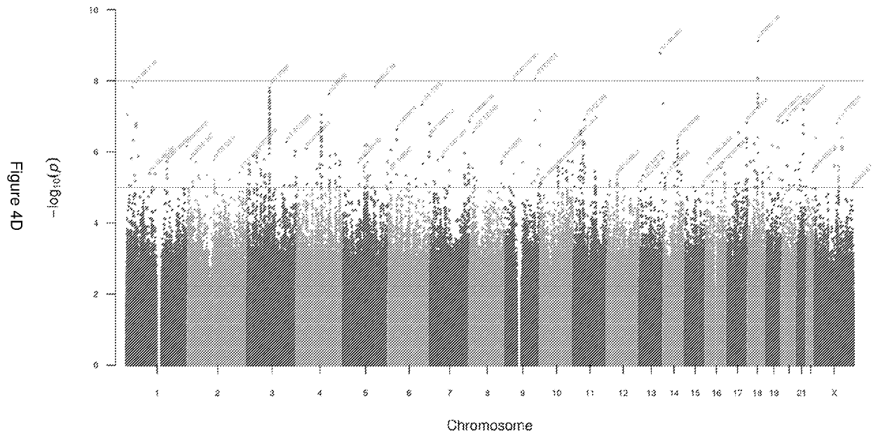

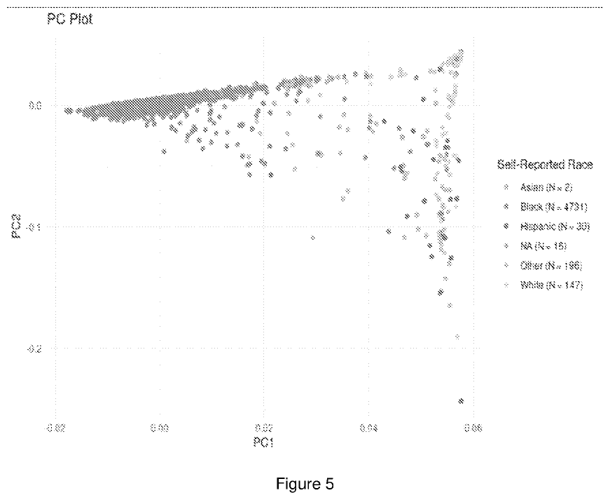

SNP-based heritability (h²) of I, L, and Q was estimated using restricted maximum likelihood (REML) as implemented in Genome-wide complex trait analysis (GCTA) version 1.92.3. Covariates included in the h² analysis included sequencing depth, batch number, maternal height, pre-pregnancy BMI, parity, self-reported race/ethnicity, and the first 10 SNP-based ancestry principal components (PCs). Self-reported race and ancestry principal components capture different aspects of genetic variation and non-genetic factors that could influence phenotypic variance in cervical length, thus both were included covariates to minimize confounding and bias (FIG. 5). SNP-based heritability was also calculated for maternal height, pre-pregnancy BMI, gestational age at delivery (GAD), and spontaneous preterm birth (sPTB). These were generated in order to compare within-cohort estimates to published SNP- and twin-based heritabilities. GCTA was also used to estimate the bivariate genetic correlation between I, L, and Q estimates and GAD/sPTB outcomes using the bivariate REML approach, which utilizes genetic variance-covariance matrices to summarize genetic relationships between a pair of traits.

### Polygenic Score Development

Polygenic scores (PGS) for I, L, and Q growth parameters were constructed using the PRS-CS software. Since no external validation cohort with both longitudinal CL data and genomic data was available, a subset of 4,302 unique women with complete genetic and demographic data was partitioned into independent training and testing sets. Approximately 80% of the sample (N=3,565) was used to train the PGSs, while 20% of the sample (N=827) was used to test the efficacy of the PGS to predict sCL. I, L, and Q were re-estimated using only data from the training sample to ensure that no data leakage occurred between the training and testing samples during the PGS analysis.

PLINK2 version v2.00a was used to test for associations between SNPs and the CL growth parameters (I, L, and Q), and gestational duration (GAD and sPTB) in the training sample. Generalized linear model (GLM) regression was used for the quantitative phenotypes (I, L, Q, and GAD), while logistic regression was used for the binary sPTB outcome phenotype. The same variance-standardized covariates used in the GCTA analysis were included in the GLM and logistic regression analyses.

The PLINK2 summary statistics from the I, L and Q genetic associations were used to estimate marginal per-allele effect sizes for the PGS analysis. African ancestry (AFR) samples from the 1000 Genomes Project Phase 3 were used as the LD reference panel and a global shrinkage parameter of phi=1e-2 was specified due to sample size. Individual-level PGSs for the 827 participants in the testing sample were then produced using the PRS-CS output and PLINK2's allelic scoring command. The accuracy with which PGSs can predict sCL was assessed via receiver operating characteristic (ROC) curves. Area under the curve (AUC) and 95% confidence intervals were estimated for each outcome. Prediction of sCL using PGS was compared to prediction of sCL using the following set of clinical predictors: maternal age, pre-pregnancy BMI, parity, number of previous preterm births, and number of previous abortions (including both spontaneous and induced abortions). Prediction of sCL in primigravida women used only maternal age and pre-pregnancy BMI as clinical predictors due to the lack of previous obstetric history for women in their first pregnancy.

### Annotation of GWAS Findings

An exploratory genome wide association study (GWAS) using data from the full cohort of N=4456 women was conducted to identify suggestive relationships between genes/biological pathways and cervical change during pregnancy. The integrative web-based platform FUMA was used to analyze and annotate variant-level summary statistics from GWASs of CL growth parameters and birth outcomes using the full cohort. A Bonferroni-corrected genome-wide significance threshold of 1.6×10⁻⁸ was used for individual SNP association tests, based on the estimated number of independent LD blocks/effective markers in African and admixed African populations. Variants with suggestive associations (1.0×10⁻⁵) were considered for exploratory gene-set enrichment analyses. Within FUMA, functional annotation and the mapping of SNPs to genes was performed using SNP2GENE, while the GENE2FUNC process was used to obtain insight into putative biological mechanisms and annotation of prioritized genes in their biological context. Tissue specific expression patterns were provided for 53 tissue types based on GTEx v8 RNA-seq data. Gene-wise associations and gene-set analysis of GWAS summary statistics were conducted with FUMA to consider the joint effect of multiple genetic markers simultaneously.

### Pre-Registration

Analyses were pre-registered with the Center for Open Science using the AsPredicted format (https://osf.io/km2cg).

### Results

### Heritability and Bivariate Genetic Correlations

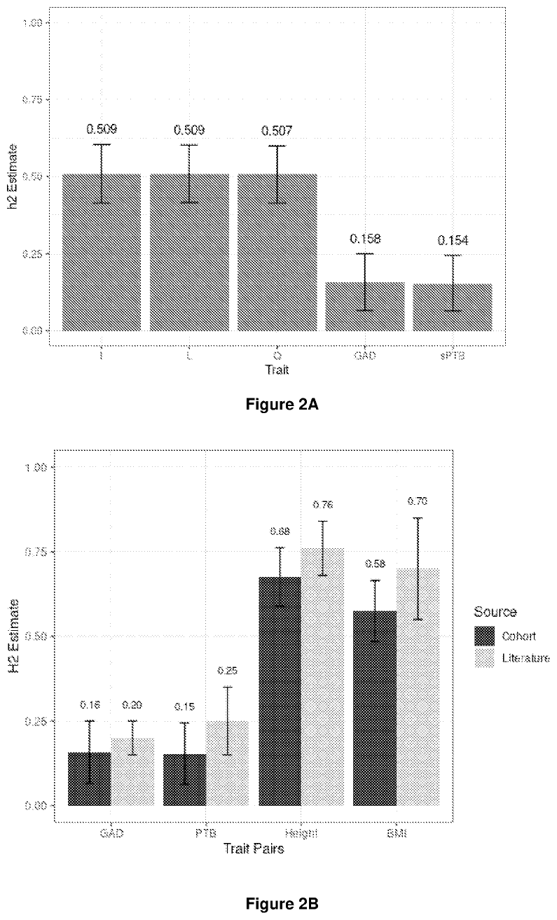

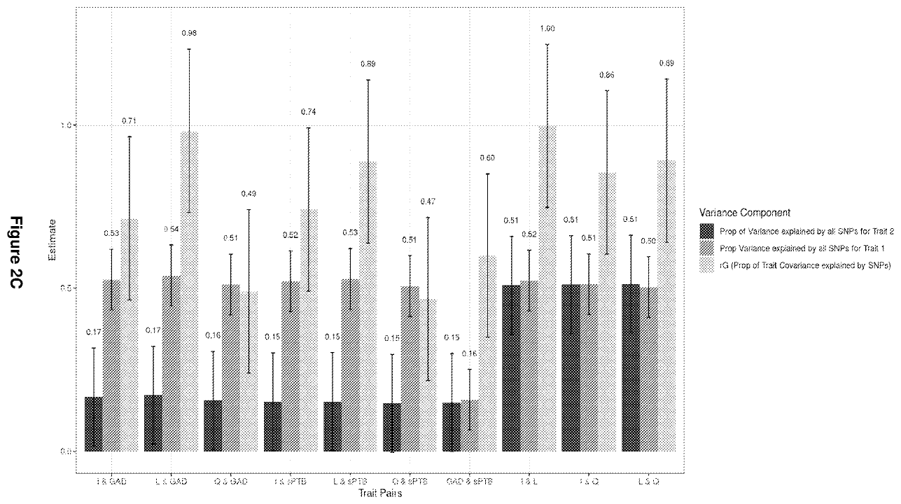

The heritability of all CL growth parameters was approximately 51% and was significantly different from zero (h²₁=0.509 (SE: 0.093); h²_{L}=0.509 (SE: 0.095); h²_{Q}=0.507 (SE: 0.093)) (FIG. 2A, Table 2). Within-cohort estimates for traits with SNP-based heritabilities previously reported in the literature (h²_{GAD}=0.158 (SE: 0.092); h²_{sPTB}=0.154 (SE: 0.091); h²_{Height}=0.675 (SE: 0.086), h²_{BMI}=0.576 (SE: 0.090)) are within the range of reported values³⁸⁻⁴⁰, which can vary by study design and population composition (FIG. 2B, Table 2). Genetic correlations (ρ_{G}) between CL growth parameters and gestational duration were significantly different than zero (ρ_{G}I-GAD=0.714 (SE: 0.229); ρ_{G}L-GAD=0.982 (SE: 0.257); ρ_{G} Q-GAD=−0.490 (SE: 0.242); ρ_{G} I-sPTB=0.741 (SE: 0.268); ρ_{G} L-sPTB=0.888 (SE: 0.284); ρ_{G} Q-sPTB=−0.467 (SE: 0.258)) (FIG. 2C, Table 3).

TABLE 2
Heritability estimates for birth outcome and biometric traits in our cohort, compared
to heritability estimates of the same traits in the published literature*.

| | | | | | | | | | Literature h² | |
|---|---|---|---|---|---|---|---|---|---|---|
| | | | | | | | | | (SNP-based | Literature |
| Trait | V_{G} | V_{G} SE | V_{E} | V_{E} SE | V_{P} | V_{P} SE | h² | h² SE | GCTA) | SE |
| I | 9.581 | 1.797 | 9.242 | 1.737 | 18.823 | 0.407 | 0.509 | 0.093 | NA | NA |
| L | 0.945 | 0.180 | 0.912 | 0.174 | 1.857 | 0.040 | 0.509 | 0.095 | NA | NA |
| Q | 0.015 | 0.003 | 0.015 | 0.003 | 0.029 | 0.001 | 0.507 | 0.093 | NA | NA |
| MTCL | 16.401 | 6.232 | 33.765 | 6.147 | 50.166 | 1.264 | 0.327 | 0.123 | NA | NA |
| GAD | 1.418 | 0.829 | 7.553 | 0.830 | 8.971 | 0.187 | 0.158 | 0.092 | 0.17 | 0.05 |
| PTB | 0.013 | 0.008 | 0.073 | 0.008 | 0.086 | 0.002 | 0.154 | 0.091 | 0.25 | 0.08 |
| Height | 0.004 | 0.000 | 0.002 | 0.000 | 0.005 | 0.000 | 0.675 | 0.086 | 0.45 | 0.04 |
| BMI | 32.918 | 5.292 | 24.228 | 5.075 | 57.146 | 1.203 | 0.576 | 0.090 | 0.22 | 0.03 |

V_{G} represents the variation from additive genetic effects, V_{E} represents variation from residual effects, Vₚ represents the total phenotypic variation, and h² (V_{G}/Vₚ) represents the proportion of genotypic to phenotypic variation and is interpreted as the proportion of variation explained for quantitative traits.
SE represents the corresponding standard error for each measurement.
*Zhang et al. N Engl J Med 2017; 377(12): 1156-67; Yengo et al. Nature 2022; 610 (7933): 704-12 and Guo et al. Sci Rep 2021; 11(1): 5240.

Table 3. (shown below as 3A and 3B) Estimates for generic correlations between cervical length growth parameters and gestational duration. Note: trait 1 represents the first trait listed in the trait name (for the correlation between 1 & GAD, I is trait 1 and GAD is trait 2). Corp represents the correlation between the phenotypic values of the two traits. V_{G} t1 and V_{G} t2 represent the variation from additive genetic effects for traits 1 and 2, respectively; C_{G}t12 represents the genetic covariance between traits 1 and 2; V_{E} t1 and V_{E} t2 represent the residual/environmental variance for traits 1 and 2, respectively; C_{E}t12 represents the residual/environmental covariance between traits 1 and 2; V_{P} t1 and V_{P} t2 represent the total phenotypic variation traits 1 and 2, respectively; h² (V_{G}/Vₚ) represents the proportion of phenotypic variance explained by all SNPs for each trait; and po represents the genetic correlation between trait 1 and 2.

TABLE 3A

| Trait | Cor_{P} | V_{G} t1 | V_{G} t2 | C_{G}t12 | V_{E} t1 | V_{E} t2 | C_{E}t12 | V_{P} t1 | V_{P} t2 |
|---|---|---|---|---|---|---|---|---|---|
| I & GAD | 0.314 | 10.018 | 1.492 | 2.759 | 9.012 | 7.480 | 0.663 | 19.030 | 8.972 |
| L & GAD | 0.510 | 1.014 | 1.544 | 1.229 | 0.866 | 7.429 | −0.090 | 1.880 | 8.973 |
| Q & GAD | −0.004 | 0.015 | 1.402 | −0.071 | 0.014 | 7.568 | −0.013 | 0.030 | 8.971 |
| I & sPTB | −0.205 | 9.861 | 0.013 | −0.266 | 9.065 | 0.073 | 0.008 | 18.926 | 0.086 |
| L & sPTB | −0.337 | 0.989 | 0.013 | −0.101 | 0.882 | 0.073 | 0.006 | 1.871 | 0.086 |
| Q & sPTB | 0.015 | 0.015 | 0.013 | 0.006 | 0.015 | 0.073 | 0.000 | 0.030 | 0.086 |
| GAD & sPTB | −0.621 | 1.419 | 0.013 | −0.082 | 7.552 | 0.074 | −0.475 | 8.971 | 0.087 |
| I & L | 0.847 | 9.856 | 0.944 | 3.040 | 8.976 | 0.913 | 1.961 | 18.832 | 1.857 |
| I & Q | −0.617 | 9.638 | 0.015 | −0.326 | 9.186 | 0.014 | −0.163 | 18.825 | 0.029 |
| L & Q | −0.434 | 0.934 | 0.015 | −0.106 | 0.922 | 0.014 | −0.044 | 1.856 | 0.030 |

TABLE 3B

| Trait | h² t1 | h² t1 SE | h² t2 | h² t2 SE | ρ_{G} | ρ_{G} SE |
|---|---|---|---|---|---|---|
| I & GAD | 0.526 | 0.093 | 0.166 | 0.092 | 0.714 | 0.229 |
| L & GAD | 0.539 | 0.094 | 0.172 | 0.091 | 0.982 | 0.257 |
| Q & GAD | 0.511 | 0.093 | 0.156 | 0.092 | −0.490 | 0.242 |
| I & sPTB | 0.521 | 0.093 | 0.152 | 0.091 | −0.741 | 0.268 |
| L & sPTB | 0.529 | 0.094 | 0.153 | 0.090 | −0.888 | 0.284 |
| Q & sPTB | 0.507 | 0.093 | 0.148 | 0.091 | 0.467 | 0.258 |
| GAD & sPTB | 0.158 | 0.092 | 0.150 | 0.091 | −0.600 | 0.262 |
| I & L | 0.523 | 0.093 | 0.508 | 0.095 | 0.997 | 0.026 |
| I & Q | 0.512 | 0.093 | 0.511 | 0.093 | −0.855 | 0.062 |
| L & Q | 0.503 | 0.094 | 0.512 | 0.092 | −0.891 | 0.065 |

### Polygenic Score Validation

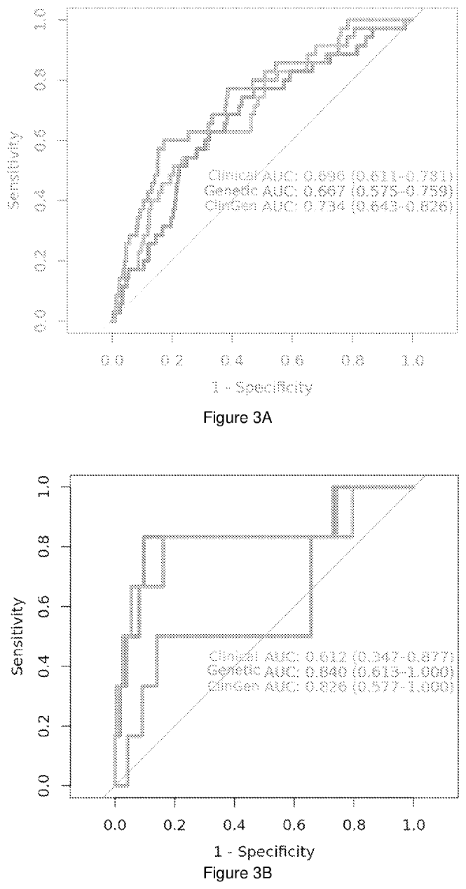

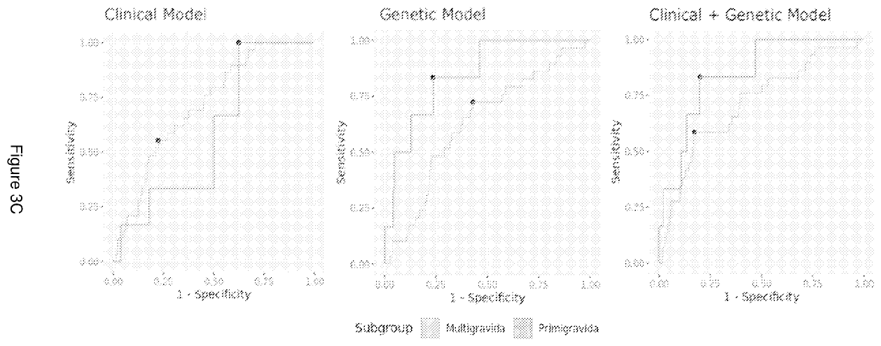

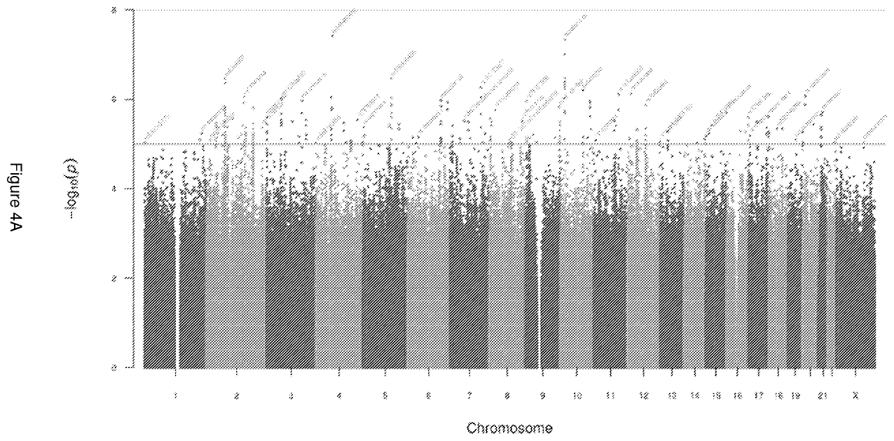

The predictive accuracy of PGSs is constrained by both the heritability and population prevalence of the outcome of interest. Based on a heritability of h_{2(sCL)}=0.51 and ~2% population prevalence of sCL, the maximum possible AUC for polygenic prediction of sCL was estimated to be approximately 0.90, while the maximum estimated AUC for polygenic prediction of sPTB was approximately 0.70 (h_{2(sPTB)}=0.15; prevalence ~10%). Prediction of sCL including PGSs for I, L, and Q (AUC_{Genetic}=0.656; 95% CI=0.562-0.751) was comparable to the predictive power of relevant clinical predictors (AUC_{Clinical}=0.693; 95% CI=0.607-0.773). Prediction of sCL was improved by combining polygenic and clinical predictors (AUC_{ClinGen}=0.727; 95% CI=0.636-0.812) (FIG. 3A). In the subset of primigravida in the training sample (N=29), inclusion of PGSs for I, L, and Q were more predictive of sCL (AUC_{Genetic}=0.820; 95% CI=0.548-0.976) and substantially outperformed clinical predictors alone (AUC_{Clinical}=0.594; 95% CI=0.368-0.823) (FIG. 3B-3C). Prediction of sCL using both polygenic and clinical predictors was comparable to using genetic predictors alone (AUC=0.818; 95% CI=0.524-0.981).

### Cervical Length GWAS Annotation

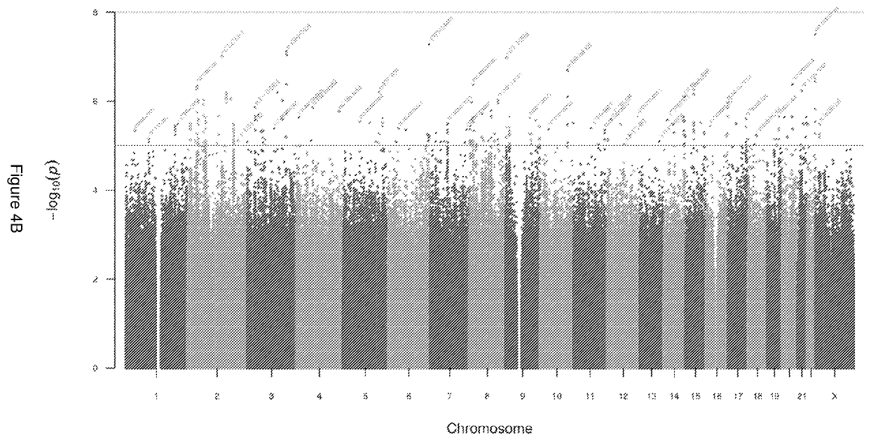

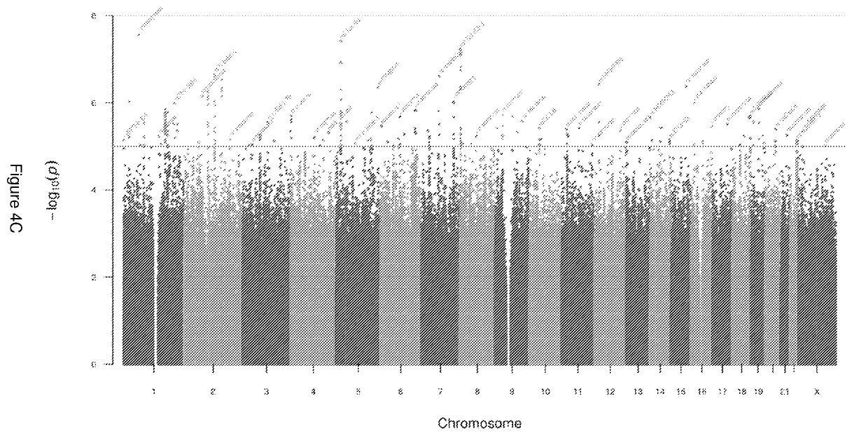

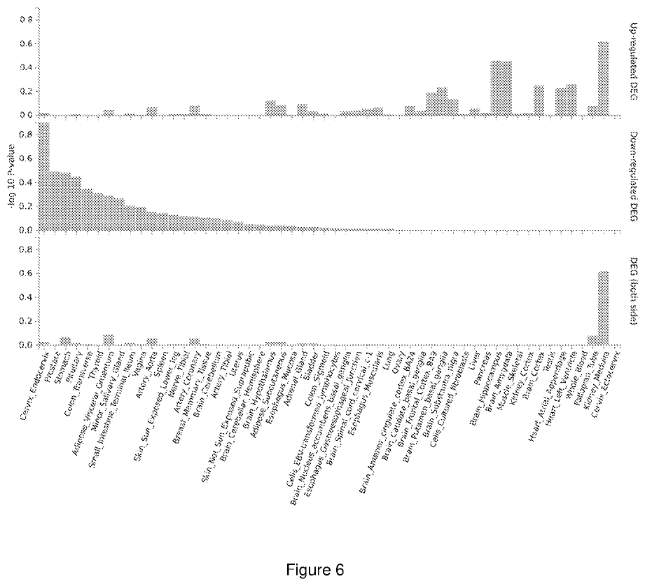

No variants associated with CL growth parameters were significant using a genome-wide significance threshold of 1.6×10⁻⁸ for the full sample (N=4456). However, 150 independent genomic loci containing 300 genes were identified across all growth parameters using a “suggestive” association threshold of 1×10⁻⁵ (FIGS. 4A-4C). Gene-set enrichment tests of all genes mapped to suggestive SNPs in either the I, L, or Q resulted in significant findings using a false discovery rate (FDR) of 5% (Tables 4-12). Several Kegg pathways (Table 8) and gene ontology pathways for biological processes (Table 9), molecular functions (Table 10), and cellular components (Table 11) were also significantly enriched. Genes mapped to suggestive hits showed downregulated expression in GTEx v8 endocervical tissue (FIG. 6).

TABLE 4
Top 10 gene-set enrichment categories identified using gene-based
tests of GWAS summary statistics for I term, computed by MAGMA.

| Gene Set | N genes | Beta | Beta STD | SE | P | P_{bon} |
|---|---|---|---|---|---|---|
| Interleukin 2 family | 55 | 0.512 | 0.0269 | 0.1174 | 6.558e−06 | 0.1015 |
| signaling | | | | | | |
| Interleukin 20 family | 26 | 0.710 | 0.0257 | 0.1657 | 9.188e−06 | 0.1423 |
| signaling | | | | | | |
| Interferon (IFN-a) cell | 10 | 0.973 | 0.0218 | 0.2367 | 1.974e−05 | 0.3057 |
| signaling | | | | | | |
| Free ubiquitin chain | 6 | 1.089 | 0.0189 | 0.2739 | 3.502e−05 | 0.5423 |
| polymerization | | | | | | |
| Receptor signaling | 150 | 0.294 | 0.0255 | 0.0740 | 3.607e−05 | 0.5584 |
| pathway via Stat | | | | | | |
| Jak Stat signaling | 148 | 0.288 | 0.0248 | 0.0758 | 7.349e−05 | 1 |
| pathway | | | | | | |
| Il2 Stat5 pathway | 30 | 0.571 | 0.0222 | 0.1540 | 1.059e−04 | 1 |
| Regulation of interferon | 25 | 0.652 | 0.0231 | 0.1821 | 1.721e−04 | 1 |
| (IFN-a) signaling | | | | | | |
| Upregulation response to | 22 | 0.649 | 0.0216 | 0.1827 | 1.912e−04 | 1 |
| selective estrogen | | | | | | |
| receptor modulators | | | | | | |
| (SERMs) or estrogen | | | | | | |
| receptor antagonists | | | | | | |
| Negative regulation of | 13 | 0.928 | 0.0237 | 0.2616 | 1.942e−04 | 1 |
| sodium ion transmembrane | | | | | | |
| transport | | | | | | |

TABLE 5
Top 10 gene-set enrichment categories identified using gene-based
tests of GWAS summary statistics for L term, computed by MAGMA.

| Gene Set | N genes | Beta | Beta STD | SE | P | P_{bon} |
|---|---|---|---|---|---|---|
| Transepithelial transport | 19 | 0.7147 | 0.0221 | 0.1988 | 1.621e−04 | 1 |
| Interleukin 2 family signaling | 55 | 0.4179 | 0.0220 | 0.1171 | 1.803e−04 | 1 |
| 5′ to 3′ exonuclease activity | 17 | 0.6532 | 0.0191 | 0.1834 | 1.854e−04 | 1 |
| Molecular function regulator | 1737 | 0.0735 | 0.0208 | 0.0215 | 3.055e−04 | 1 |
| ATF2 signaling pathways | 57 | 0.3946 | 0.0211 | 0.1164 | 3.499e−04 | 1 |
| Negative regulation of histone | 6 | 0.9501 | 0.0165 | 0.2839 | 4.101e−04 | 1 |
| h3 k9 trimethylation | | | | | | |
| Rho gtpases activate rhotekin | 9 | 0.8175 | 0.0174 | 0.2451 | 4.259e−04 | 1 |
| and rhophilins | | | | | | |
| Interleukin 20 family | 26 | 0.5468 | 0.0198 | 0.1654 | 4.738e−04 | 1 |
| signaling | | | | | | |
| VEGFA targets | 98 | 0.2965 | 0.0208 | 0.0899 | 4.877e−04 | 1 |
| Protein localization to | 8 | 0.9918 | 0.0199 | 0.3025 | 5.214e−04 | 1 |
| photoreceptor outer segment | | | | | | |

TABLE 6
Top 10 gene-set enrichment categories identified using gene-based
tests of GWAS summary statistics for Q term, computed by MAGMA.

| Gene Set | N genes | Beta | Beta STD | SE | P | P_{bon} |
|---|---|---|---|---|---|---|
| E-box enhancer binding | 49 | 0.4677 | 0.0232 | 0.1225 | 6.773e−05 | 1 |
| Positive regulation of integrin | 8 | 1.1064 | 0.0222 | 0.3059 | 1.496e−04 | 1 |
| activation | | | | | | |
| Lipid phosphorylation | 62 | 0.3824 | 0.0213 | 0.1061 | 1.572e−04 | 1 |
| Interleukin 20 family | 26 | 0.5879 | 0.0213 | 0.1654 | 1.903e−04 | 1 |
| signaling | | | | | | |
| Forebrain radial glial cell | 7 | 1.1321 | 0.0213 | 0.3273 | 2.720e−04 | 1 |
| differentiation | | | | | | |
| Cell migration in hindbrain | 15 | 0.7572 | 0.0208 | 0.2191 | 2.745e−04 | 1 |
| Long chain fatty acid import | 15 | 0.5946 | 0.0163 | 0.1741 | 3.199e−04 | 1 |
| into cell | | | | | | |
| Receptor complex | 389 | 0.1527 | 0.0212 | 0.0451 | 3.563e−04 | 1 |
| Insulin signaling pathway | 136 | 0.2495 | 0.0206 | 0.0738 | 3.600e−04 | 1 |
| Down-regulated after | 438 | 0.1345 | 0.0198 | 0.0403 | 4.256e−04 | 1 |
| stimulation with galecin-1 | | | | | | |
| (lectin, LGALS1) vs | | | | | | |
| lipopolysaccharide (LPS). | | | | | | |

TABLE 7
Positional gene enrichment sets, using summary statistics from I, L, and Q GWASs.

| Gene Set | N | n | P-value | Adj P | Genes |
|---|---|---|---|---|---|
| chr17q25 | 263 | 53 | 4.93e−46 | 1.47e−43 | RPL38, TTYH2, DNAI2, RNA5SP448, GPRC5C, |
| | | | | | CD300A, CD300LB, CD300LD, CD300E, RAB37, |
| | | | | | MIR3615, MIR3615, SLC9A3R1, TMEM104, |
| | | | | | GRIN2C, FDXR, FADS6, USH1G, OTOP2, OTOP3, |
| | | | | | HID1, RP11-309N17.4, CDR2L, ICT1, RNU6-362P, |
| | | | | | KCTD2, ATP5H, RN7SL573P, SLC16A5, ARMC7, |
| | | | | | NT5C, HN1, SUMO2, NUP85, GGA3, MRPS7, |
| | | | | | MIF4GD, SLC25A19, GRB2, MIR3678, RNU6-938P, |
| | | | | | KIAA0195, CASKIN2, TSEN54, LLGL2, MYO15B, |
| | | | | | RECQL5, SAP30BP, SRP68, ZACN, GALR2, |
| | | | | | EXOC7, RNF157, FOXK2 |
| chr2p13 | 121 | 27 | 3.28e−25 | 4.90e−23 | SLC4A5, KRT18P26, RNU6-542P, NECAP1P2, |
| | | | | | TAF13P2, DCTN1, DCTN1-AS1, HMGA1P8, |
| | | | | | WDR54, RTKN, INO80B, WBP1, MOGS, MRPL53, |
| | | | | | CCDC142, TTC31, LBX2, LBX2-AS1, PCGF1, TLX2, |
| | | | | | DQX1, AUP1, HTRA2, LOXL3, M1AP, TVP23BP2, |
| | | | | | SEMA4F |
| chr1q32 | 224 | 16 | 7.98e−8 | 7.95e−6 | RNU6-778P, NR5A2, RNU6-570P, SNORA70, IL19, |
| | | | | | IL20, KCNH1, RCOR3, TRAF5, LINC00467, |
| | | | | | SNX25P1, RD3, RP11-552D8.1, INTS7, RP11- |
| | | | | | 15I11.3, TMEM206 |
| chr14q24 | 168 | 10 | 1.02e−4 | 7.65e−3 | DCAF5, COX16, SYNJ2BP-COX16, RNU2-51P, |
| | | | | | SYNJ2BP, ADAM20P1, RNU6-659P, MED6, |
| | | | | | KRT18P7, RN7SL77P |
| chr3p13 | 37 | 5 | 1.33e−4 | 7.98e−3 | RNA5SP136, GXYLT2, PPP4R2, RNU7-19P, EBLN2 |
| chr7q34 | 148 | 9 | 1.92e−4 | 9.58e−3 | SSBP1, TAS2R3, TAS2R4, MTND2P5, MTND1P3, |
| | | | | | MYL6P4, OR9A1P, MGAM, TAS2R38 |
| chr3q23 | 50 | 5 | 5.62e−4 | 2.40e−2 | XRN1, RNU1-100P, ATR, PLS1, TRPC1 |
| chr8q11 | 52 | 5 | 6.74e−4 | 2.52e−2 | MAPK6PS4, ATP6V1G1P2, IGLV8OR8-1, SPIDR, |
| | | | | | SNTG1 |
| chr1p22 | 111 | 7 | 7.91e−4 | 2.63e−2 | MCOLN2, WDR63, MCOLN3, U3, FAM69A, RNU6- |
| | | | | | 970P, RN7SL692P |
| chr14q32 | 501 | 16 | 1.44e−3 | 4.30e−2 | SETD3, CCNK, CCDC85C, MIR496, MIR377, |
| | | | | | MIR541, MIR409, MIR412, MIR369, MIR410, |
| | | | | | MIR656, MEG9, DYNC1H1, RCOR1, TRAF3, AMN |

TABLE 8
Kegg Pathway Analysis for I, L, and Q mapped genes

| Gene Set | N | n | P-value | Adj P | Genes |
|---|---|---|---|---|---|
| Long Term | 70 | 6 | 3.69e−4 | 4.42e−2 | GRIN2C, PRKCG, |
| Potentiation | | | | | CAMK2D, GRIA2, |
| | | | | | ITPR3, ADCY8 |
| Taste | 51 | 5 | 6.16e−4 | 4.42e−2 | ITPR3, TAS2R3, |
| Transduction | | | | | TAS2R4, TAS2R38, |
| | | | | | ADCY8 |
| Calcium | 177 | 9 | 7.13e−4 | 4.42e−2 | GRIN2C, PRKCG, |
| Signaling | | | | | SLC8A1, TNNC2, |
| Pathway | | | | | TRPC1, CAMK2D, |
| | | | | | ITPR3, ADCY8, |
| | | | | | GRPR |

TABLE 9
Gene Ontology: Biological Processes for I, L, and Q mapped genes

| Gene Set | N | n | P-value | Adj P | Genes |
|---|---|---|---|---|---|
| Cellular | 1886 | 50 | 4.64e−6 | 3.30e−2 | ATG4C, WLS, DNM3, RAB4A, |
| Macromolecule | | | | | NRP1, TUB, DLG2, RAB38, |
| Localization | | | | | KPNA3, PRKCH, SYNJ2BP, AMN, |
| | | | | | AP3B2, BANP, RPL38, RAB37, |
| | | | | | SLC9A3R1, GRIN2C, HID1, NUP85, |
| | | | | | GGA3, SRP68, CDC37, DNM2, |
| | | | | | TMED1, CARM1, MARK4, KPTN, |
| | | | | | NAPA, MYADM, DDX1, |
| | | | | | COMMD1, RTKN, AUP1, HTRA2, |
| | | | | | SNAP25-AS1, SRSF6, ACOT8, |
| | | | | | MRAP, ATR, PLS1, TSPAN5, |
| | | | | | LEMD2, RIMS1, NUPL2, HIP1, |
| | | | | | CNTNAP2, MCPH1, SPIDR, APBA1 |
| Regulation Of | 475 | 20 | 8.97e−6 | 3.30e−2 | DNM3, NRP1, PLXNC1, |
| Cell | | | | | SLC9A3R1, RNF157, DNM2, |
| Morphogenesis | | | | | MYADM, TLX2, SEMA4F, RTN4R, |
| | | | | | PARVB, LIMD1, EFNA5, RIMS1, |
| | | | | | HECW1, DLC1, DOCK5, PTPRD, |
| | | | | | PALM2, PAK3 |

TABLE 10
Gene Ontology: Molecular Functions for I, L, and Q mapped genes

| Gene Set | N | n | P-value | Adj P | Genes |
|---|---|---|---|---|---|
| Haptoglobin | 10 | 4 | 6.44e−6 | 1.06e−2 | HBM, HBA2, HBA1, HBQ1 |
| Binding | | | | | |
| Molecular | 41 | 6 | 1.76e−5 | 1.45e−2 | KPNA3, HBM, HBA2, HBA1, HBQ1, |
| Carrier | | | | | NUPL2 |
| Activity | | | | | |
| Oxygen | 14 | 4 | 2.94e−5 | 1.61e−2 | HBM, HBA2, HBA1, HBQ1 |
| Carrier | | | | | |
| Activity | | | | | |
| Heparin | 163 | 10 | 7.96e−5 | 3.27e−2 | NRP1, NAV2, BSPH1, ELSPBP1, |
| Binding | | | | | MSTN, RTN4R, ANXA6, LPA, |
| | | | | | GPNMB, LPL |
| Oxygen | 36 | 5 | 1.17e−4 | 3.84e−2 | HBM, HBA2, HBA1, HBQ1, ADGB |
| Binding | | | | | |

TABLE 11
Gene Ontology: Cellular Components for I, L, and Q mapped genes

| Gene Set | N | n | P-value | Adj P | Genes |
|---|---|---|---|---|---|
| Synaptic | 428 | 19 | 7.14e−6 | 3.80e−3 | DNM3, KCNH1, NRP1, DLG2, |
| Membrane | | | | | CNTN5, KCNC2, SYNJ2BP, |
| | | | | | GRIN2C, DNM2, PRKCG, SLC8A1, |
| | | | | | SEMA4F, LRRTM4, GRIA2, RIMS1, |
| | | | | | UTRN, HIP1, ADCY8, APBA1 |
| Haptoglobin | 11 | 4 | 1.00e−5 | 3.80e−3 | HBM, HBA2, HBA1, HBQ1 |
| Hemoglobin | | | | | |
| Complex | | | | | |
| Hemoglobin | 12 | 4 | 1.48e−5 | 3.80e−3 | HBM, HBA2, HBA1, HBQ1 |
| Complex | | | | | |
| Plasma Membrane | 1185 | 35 | 1.52e−5 | 3.80e−3 | WLS, DNM3, KCNH1, DISP1, NRP1, |
| Region | | | | | PLEKHA1, DLG2, CNTN5, KCNC2, |
| | | | | | SYNJ2BP, AMN, SLC9A3R1, |
| | | | | | GRIN2C, PDE4A, DNM2, PRKCG, |
| | | | | | SLC8A1, SLC4A5, SEMA4F, |
| | | | | | LRRTM4, PARD3B, SLC6A20, |
| | | | | | GRIA2, EFNA5, RIMS1, UTRN, |
| | | | | | HIP1, TAS2R4, MGAM, CNTNAP2, |
| | | | | | DLC1, SNTG1, ADCY8, APBA1, |
| | | | | | NHS |
| Cortical | 110 | 9 | 1.91e−5 | 3.82e−3 | LLGL2, MYADM, RTKN, GYPC, |
| Cytoskeleton | | | | | PLS1, RIMS1, UTRN, HIP1, DLC1 |
| Cell Cortex Part | 176 | 11 | 2.98e−5 | 4.54e−3 | LLGL2, EXOC7, MYADM, DCTN1, |
| | | | | | RTKN, GYPC, PLS1, RIMS1, UTRN, |
| | | | | | HIP1, DLC1 |
| Postsynapse | 610 | 22 | 3.52e−5 | 4.54e−3 | DNM3, KCNH1, RAB4A, NRP1, |
| | | | | | DLG2, KCNC2, SYNJ2BP, RPL38, |
| | | | | | GRIN2C, DNM2, KPTN, NAPA, |
| | | | | | PRKCG, SLC8A1, SEMA4F, |
| | | | | | LRRTM4, GRIA2, UTRN, HIP1, |
| | | | | | ADCY8, APBA1, PAK3 |
| Postsynaptic | 321 | 15 | 3.63e−5 | 4.54e−3 | DNM3, KCNH1, NRP1, DLG2, |
| Membrane | | | | | KCNC2, SYNJ2BP, GRIN2C, DNM2, |
| | | | | | SLC8A1, SEMA4F, LRRTM4, |
| | | | | | GRIA2, UTRN, HIP1, ADCY8 |
| Postsynaptic | 8 | 3 | 1.30e−4 | 1.15e−2 | DNM3, DNM2, GRIA2 |
| Endocytic Zone | | | | | |
| Cortical Actin | 83 | 7 | 1.33e−4 | 1.15e−2 | LLGL2, MYADM, RTKN, PLS1, |
| Cytoskeleton | | | | | UTRN, HIP1, DLC1 |
| Cytoplasmic | 488 | 18 | 1.35e−4 | 1.15e−2 | WLS, DLG2, RPGRIP1, FAM154B, |
| Region | | | | | AP3B2, DNAI2, LLGL2, EXOC7, |
| | | | | | MYADM, DCTN1, RTKN, GYPC, |
| | | | | | PARD3B, PLS1, RIMS1, UTRN, |
| | | | | | HIP1, DLC1 |
| Actin Cytoskeleton | 491 | 18 | 1.45e−4 | 1.15e−2 | HNRNPC, SLC9A3R1, USH1G, |
| | | | | | LLGL2, FHOD3, KPTN, MYADM, |
| | | | | | DCTN1, RTKN, TNNC2, PARVB, |
| | | | | | XIRP1, PLS1, PALLD, UTRN, HIP1, |
| | | | | | DLC1, ADCY8 |
| Neuron Part | 1709 | 42 | 1.49e−4 | 1.15e−2 | WLS, DNM3, KCNH1, RAB4A, |
| | | | | | NRP1, ARMS2, DLG2, CNTN5, |
| | | | | | KCNC2, RPGRIP1, SYNJ2BP, |
| | | | | | AP3B2, RPL38, SLC9A3R1, |
| | | | | | GRIN2C, USH1G, ZACN, EXOC7, |
| | | | | | DNM2, SMARCA4, MARK4, KLC3, |
| | | | | | KPTN, NAPA, PRKCG, SLC8A1, |
| | | | | | DCTN1, NCAM2, RTN4R, XRN1, |
| | | | | | RGS12, CAMK2D, GRIA2, PALLD, |
| | | | | | ITPR3, RIMS1, UTRN, HIP1, |
| | | | | | CNTNAP2, ADCY8, APBA1, PAK3 |
| Cd40 Receptor | 11 | 3 | 3.71e−4 | 2.39e−2 | TRAF5, TRAF3, HTRA2 |
| Complex | | | | | |
| Cell Leading Edge | 397 | 15 | 3.72e−4 | 2.39e−2 | WLS, KCNH1, PLEKHA1, KCNC2, |
| | | | | | SLC9A3R1, PDE4A, DNM2, KPTN, |
| | | | | | MYADM, PARVB, PALLD, |
| | | | | | CNTNAP2, DLC1, SNTG1, NHS |
| Receptor Complex | 398 | 15 | 3.82e−4 | 2.39e−2 | TRAF5, NRP1, DLG2, PLXNC1, |
| | | | | | SYNJ2BP, TRAF3, GPRC5C, |
| | | | | | GRIN2C, HTRA2, LRP1B, CALCRL, |
| | | | | | SACM1L, TRPC1, GRIA2, ITPR3 |
| Leading Edge | 170 | 9 | 5.34e−4 | 3.14e−2 | WLS, KCNH1, PLEKHA1, KCNC2, |
| Membrane | | | | | PDE4A, DNM2, CNTNAP2, DLC1, |
| | | | | | SNTG1 |
| Synapse | 1169 | 30 | 6.65e−4 | 3.70e−2 | DNM3, KCNH1, RAB4A, NRP1, |
| | | | | | DLG2, CNTN5, KCNC2, SYNJ2BP, |
| | | | | | RPL38, GRIN2C, ZACN, DNM2, |
| | | | | | KPTN, NAPA, PRKCG, SLC8A1, |
| | | | | | SEMA4F, LRRTM4, RTN4R, XRN1, |
| | | | | | RGS12, GRIA2, EFNA5, RIMS1, |
| | | | | | UTRN, HIP1, ADCY8, PTPRD, |
| | | | | | APBA1, PAK3 |
| Neurotransmitter | 54 | 5 | 8.03e−4 | 4.13e−2 | DLG2, SYNJ2BP, GRIN2C, |
| Receptor Complex | | | | | SACM1L, GRIA2 |
| Cell Projection | 341 | 13 | 8.26e−4 | 4.13e−2 | WLS, KCNH1, PLEKHA1, KCNC2, |
| Membrane | | | | | AMN, SLC9A3R1, PDE4A, DNM2, |
| | | | | | UTRN, TAS2R4, CNTNAP2, DLC1, |
| | | | | | SNTG1 |
| Cell Cortex | 302 | 12 | 9.08e−4 | 4.33e−2 | LLGL2, EXOC7, MYADM, DCTN1, |
| | | | | | RTKN, GYPC, PARD3B, PLS1, |
| | | | | | RIMS1, UTRN, HIP1, DLC1 |
| Axolemma | 15 | 3 | 9.83e−4 | 4.36e−2 | KCNH1, KCNC2, CNTNAP2 |
| Synapse Part | 932 | 25 | 1.00e−3 | 4.36e−2 | DNM3, KCNH1, RAB4A, NRP1, |
| | | | | | DLG2, CNTN5, KCNC2, SYNJ2BP, |
| | | | | | RPL38, GRIN2C, DNM2, KPTN, |
| | | | | | NAPA, PRKCG, SLC8A1, SEMA4F, |
| | | | | | LRRTM4, RTN4R, GRIA2, RIMS1, |
| | | | | | UTRN, HIP1, ADCY8, APBA1, |
| | | | | | PAK3 |
| Plasma Membrane | 187 | 9 | 1.05e−3 | 4.38e−2 | TRAF5, DLG2, SYNJ2BP, TRAF3, |
| Receptor Complex | | | | | GRIN2C, HTRA2, CALCRL, |
| | | | | | SACM1L, GRIA2 |
| Cell Projection | 1438 | 34 | 1.22e−3 | 4.89e−2 | WLS, DNM3, KCNH1, NRP1, |
| Part | | | | | PLEKHA1, DLG2, KCNC2, |
| | | | | | RPGRIP1, AMN, FAM154B, AP3B2, |
| | | | | | DNAI2, SLC9A3R1, USH1G, |
| | | | | | EXOC7, PDE4A, DNM2, MARK4, |
| | | | | | KLC3, PRKCG, SLC8A1, DCTN1, |
| | | | | | RTN4R, XRN1, RGS12, GRIA2, |
| | | | | | PALLD, UTRN, TAS2R4, |
| | | | | | CNTNAP2, DLC1, SNTG1, ADCY8, |
| | | | | | APBA1 |

TABLE 12
Gene-set enrichment categories identified using gene-based tests
of GWAS summary statistics from I, L and Q, computed by MAGMA.

| Gene Set | N | n | P-value | Adj P | Genes |
|---|---|---|---|---|---|
| Creighton | 527 | 26 | 1.36e−5 | 3.24e−2 | DAK, FADS3, CDK2, WDR89, SGPP1, |
| Endocrine | | | | | ABAT, C16orf45, CRISPLD2, BCL2, |
| Therapy | | | | | ZNF121, RPS9, SOGA1, ANKRD28, |
| Resistance | | | | | LRRFIP2, RSRC1, MB21D2, |
| | | | | | ARHGAP24, GRIA2, ADAMTS19, |
| | | | | | ZBTB2, RMND1, C6orf211, ESR1, |
| | | | | | LMBR1, SLC7A2, TMEM164 |
| Downregulation | 1057 | 41 | 1.96e−5 | 3.24e−2 | NIPAL3, LRRC42, TNR, SEC16B, |
| of | | | | | LRRC4C, MYRF, IFFO1, CHD4, |
| Oligodendrocyte | | | | | PRICKLE1, DGKA, PMEL, LRRC49, |
| Differentiation | | | | | C16orf45, ARHGAP23, MPP2, |
| | | | | | SAP30BP, CYTH1, DCC, CERS6, |
| | | | | | SOGA1, NCAM2, CHADL, HACL1, |
| | | | | | ANKRD28, GOLGA4, ZBTB20, |
| | | | | | LSAMP, MB21D2, ATP8A1, |
| | | | | | RASGEF1B, DNAJB14, TRIM2, |
| | | | | | GRIA2, SNORA74, AGPAT4, |
| | | | | | MKRN1, AGK, TMEM71, NDRG1, |
| | | | | | GLIS3, FOCAD |
| Synaptic | 709 | 32 | 8.91e−6 | 4.39e−2 | TNR, GRID1, LRRC4C, SYT7, GRM5, |
| Signaling | | | | | VAMP1, FGF14, SLC8A3, GABRG3, |
| | | | | | TMOD2, ABAT, ASIC2, MPP2, |
| | | | | | DLGAP1, DCC, CACNG8, PDYN, |
| | | | | | BCR, SHISA8, CNTN4, SYN2, ERC2, |
| | | | | | FGF12, GRIA2, MCTP1, PAIP2, |
| | | | | | RIMS1, AKAP12, PARK2, DGKI, |
| | | | | | ADRA1A, CACNA1B |
| Regulation of | 430 | 23 | 1.19e−5 | 4.39e−2 | TNR, GRID1, LRRC4C, SYT7, GRM5, |
| Trans Synaptic | | | | | VAMP1, FGF14, SLC8A3, ABAT, |
| Signaling | | | | | MPP2, DCC, CACNG8, BCR, SHISA8, |
| | | | | | CNTN4, MCTP1, PAIP2, RIMS1, |
| | | | | | AKAP12, PARK2, DGKI, ADRA1A, |
| | | | | | CACNA1B |

TABLE 13
Gene-set enrichment categories identified using gene-based tests
of GWAS summary statistics for GAD, computed by MAGMA.

| Gene Set | N genes | Beta | Beta STD | SE | P | P_{bon} |
|---|---|---|---|---|---|---|
| Neuronal Dense Core | 6 | 1.5721 | 0.0273 | 0.3651 | 8.373e−06 | 0.1297 |
| Vesicle | | | | | | |
| Formation of Tubulin | 26 | 0.5853 | 0.0212 | 0.1449 | 2.683e−05 | 0.4155 |
| Folding Intermediates | | | | | | |
| Filamin Binding | 13 | 0.8736 | 0.0223 | 0.2316 | 8.120e−05 | 1 |
| Fu Interact with | 13 | 0.7143 | 0.0183 | 0.1899 | 8.473e−05 | 1 |
| Alkbh8 | | | | | | |
| Positive Regulation | 15 | 0.6390 | 0.0176 | 0.1797 | 1.888e−04 | 1 |
| of Telomerase | | | | | | |
| RNA Localization to | | | | | | |
| Cajal Body | | | | | | |
| Secondary Metabolite | 27 | 0.5598 | 0.0206 | 0.1602 | 2.379e−04 | 1 |
| Biosynthetic Process | | | | | | |
| Upregulation: Aging | 5 | 1.4413 | 0.0229 | 0.4139 | 2.495e−04 | 1 |
| Transport of | 20 | 0.6437 | 0.0204 | 0.1850 | 2.513e−04 | 1 |
| Connexons to the | | | | | | |
| Plasma Membrane | | | | | | |
| D4-GDI (GDP | 12 | 0.9218 | 0.0227 | 0.2705 | 3.274e−04 | 1 |
| dissociation inhibitor) | | | | | | |
| Pathway | | | | | | |
| H3k4me3 and H3k27me3 | 131 | 0.2536 | 0.0205 | 0.0748 | 3.499e−04 | 1 |

### Gestational Age at Delivery (GAD) GWAS Annotation

Two variants were found to be significantly associated with GAD using a genome-wide significance threshold of 1.6×10⁻⁸. These were: rs75296056 and rs 185443461 on Chromosome 18 (p=1.85×10⁻⁹) (FIG. 4D). This region is near the gene SETBP1 (SET binding protein 1), which is highly expressed in cervical tissue, and interacts with the estradiol pathway. Additionally, 4410 candidate SNPs, 400 independent lead SNPs, 329 genomic risk loci, and 950 mapped genes were identified using a “suggestive” association 10 threshold of 1×10⁻⁵ (FIG. 4D). Several curated gene sets from gene-based enrichment testing were identified, although none passed a Bonferroni correction for multiple testing (see Table 13).

### Sequencing and Imputation

Purified genomic DNA (gDNA) was extracted from 5,116 buffy coat samples and diluted to a concentration of 10 ng/μL. One microgram (μg) of gDNA was aliquoted into plates and submitted to BGI Group Next Generation Sequencing Services for low-pass, whole genome sequencing. Library prep was performed via DNA nanoball sequencing (DNBseq) using paired-end 100-bp reads on the BGISEQ-500 sequencing instrument. Of the 5,116 samples sent for sequencing, 92 samples were sequenced at 4× depth as a pilot sample, and 5,024 samples were sequenced at 1× depth. Gencove, Inc. performed sequence alignment using the GRCh37-hg19—human reference genome assembly (2009). The 1000 Genomes Phase 3, including normalized multiallelic sites and the X chromosome, was used for the imputation reference panel for the Ioimpute v0.18 imputation algorithm for low-pass sequencing data (Li et al, 2020).

### Quality Control Measures

After sequencing, 5116 unique samples from 5023 unique patients were identified. 128 pairs of over-related samples were identified; 69 pairs of samples were determined to be duplicates/from the same patient, and 421 additional pairs were determined to be from relatives (3^{rd} degree relatives or closer). Additionally, 5 samples were excluded due to poor sample/genotype data quality and 243 samples were excluded due to reduced bias due to population stratification. The patients were empirically assigned to a continental ancestry subpopulation via principal component analysis (PCA) in Plink (PLINK v2.00a3.3 LM 64-bit Intel) (Purcell, 2007) using external reference panel of known ancestry reference (1000 Genomes Project, Phase 3). 4,474 samples were inferred to belong to the African ancestry superpopulation and were included in the genetic analysis pipeline, while 142 samples from other ancestry/population backgrounds were excluded from the genetic analysis (FIG. 5). Because only a small number of samples were assigned to the non-African superpopulation cluster, and were distributed among European, Asian, and admixed clusters, we did not proceed with analyzing ancestries separately and combining via meta-analysis.

Additional exclusion of samples from the analysis was based on the presence/absence of phenotypic data. 368 patients were excluded from the current analysis for interventions during pregnancy (345 for progesterone and 23 for cerclage), and 15 samples were excluded because they could not be matched to patient identifiers from the original cohort, and. In total, 4396 samples passed all quality control measures and contained the relevant phenotypic data for inclusion in the genetic heritability analysis. After imputation, each sample had genetic variant information at 65 million genomic locations, also known as single nucleotide polymorphisms (SNPs). Variants with a genotyping rate of less than 5 percent (geno<0.05), a MAF of less than half a percent (MAF<0.005), or found to be in extreme deviation from Hardy-Weinberg equilibrium (HWE<0.000001) were excluded from the analysis. Approximately 16 million variants/SNPs remained after QC.

Although the genome-wide significance p-value threshold of 5×10⁻⁸ has become a standard for common-variant GWAS, a more stringent threshold for significant associations is needed to account for the lower allele frequency spectrum used in low-pass sequencing studies and the shorter extent of linkage disequilibrium (LD) in African and admixed African populations. The estimated number of independent LD blocks/effective markers in African populations is estimated to be approximately 3 million (compared to ~1.6 million in European populations), which translates to a Bonferroni-corrected genome-wide significance threshold of 1.6×10⁻⁸ (Li et al. Hum Genet 2012; 131 (5): 747-56). However, since this study is underpowered to identify individual variants associated with phenotypes, variants with “suggestive” associations are also considered (p<1×10⁻⁵).

A polygenic risk score (PRS) was developed using the PRS-CS software, a polygenic prediction method that infers posterior effect sizes of single nucleotide polymorphisms (SNPs) using genome-wide association summary statistics and an external linkage disequilibrium (LD) reference panel (Ge et al. Nat Commun 2019; 10 (1): 1776). PRS-CS utilizes a high-dimensional Bayesian regression framework to place a continuous shrinkage prior to SNP effect sizes. LD reference panels were constructed using the AFR samples from the 1000 Genomes Project, phase 3).

An 80/20 split was used for training and testing the PRS (trained on participants and tested on 827 participants). Variant-level summary statistics from GWASs of I, L and Q in the 3,565 participants in the training sample were used to build the PRS. PRS-CS was used to estimate a posterior SNP effect size for each SNP. A global shrinkage parameter of phi=1e-2 was specified due to the limited sample size. Individual-level polygenic scores for the 827 participants in the independent testing sample were then produced using the PRScs output and PLINK2's allelic scoring command.

### Supplemental Findings

Variance explained by Polygenic Scores

Polygenic profiling was assessed based on the proportion of explained variance in the quantitative clinical trait of MTCL and classification of binary clinical outcome of sCL (cervical length <25 mm by 24 weeks gestation). Together, the PGSs for I, L, and Q explained up to 5% of the variance in MTCL in the testing sample. Additional variance in MTCL was accounted for by including clinical predictors, including maternal age, BMI, and previous pregnancy outcomes (sPTB or previous miscarriages/terminations), which on their own explain approximately 3% of the variance in MTCL in the testing dataset. A predictive model combining PGSs and clinical risk factors explained up to 9% of the variance in MTCL in the testing sample. Odds ratios for the clinical outcomes of sCL and sPTB were increased in women with medium and high PGSs for I, L, and Q; however, these were not found to be statistically significant.

### DISCUSSION

The methods disclosed herein provide the first assessment of the genetic architecture for CL and its genetic relationship to gestational duration. Low-pass sequencing experiments and polygenic score analysis allowed for the estimation of the number, effect size, and population frequency of genetic variants essential for heritability studies of CL. The estimated heritability of ~51% for CL parameters while detecting few genome-wide significant loci is consistent with a highly polygenic and complex trait significantly influenced by both genetic and environmental factors. The robust genetic correlations observed between I, L, Q and GAD/sPTB (46%-98%) demonstrate a substantial shared genetic liability between these traits. For example, genetic factors influencing initial cervical length and accelerated shortening (increased slope) across pregnancy contribute to shorter gestational duration and an increase in risk for spontaneous preterm delivery.

Establishing the shared genetic risk between these pregnancy outcomes improves the utility of CL as a screening tool for preterm birth and encourages clinicians to consider broader screening programs to identify women with a sCL during pregnancy. A PGS for sCL allows clinicians to identify primigravida women who do not have an obstetric history to determine their risk of developing a sCL or delivering preterm. This is particularly a benefit to individuals who may be less likely to receive mid-trimester CL screening and who could benefit from more frequent CL screening during pregnancy. The method of the invention allows earlier initiation of effective clinical interventions, such as vaginal progesterone supplementation, cerclage and other treatments to reduce the risk of preterm birth in women who develop a sCL.

Exploratory gene set enrichment and pathway analysis of GWAS results shed light on biological mechanisms involved in cervical changes during pregnancy and gestational duration. Several pathways related to inflammation, tissue remodeling, and metalloproteinase and peptidase activities were identified, which have been previously implicated in the onset of labor in both premature and term birth. Genomic signals near genes in insulin, progesterone, and estrogen signaling pathways were also seen. These results align with what is known about hormonal influences and responses in cervical tissue and offer a mechanistic explanation for why intervention with vaginal progesterone helps prolong gestation and prevent preterm birth in women with a sCL. They also provide new avenues for drug discovery for preterm birth, leveraging existing, safety-tested drugs from endocrinology.

Previous genetic studies of preterm birth have focused on individuals of European ancestry in Scandinavia and the Americas, limiting the applicability of their findings across diverse global populations. This data is from the first large genomic study of birth outcomes in a large sample of US-based women with African ancestry, who are both underrepresented in genomic studies, and suffer disproportionately higher rates of sCL and sPTB. Conducting this research in a high-risk and underrepresented population contributes to more effective, personalized prevention strategies for patients from a similar genetic and sociodemographic background. The estimated PGSs improved prediction of sCL over clinical risk factors alone, and greatly outperformed the included clinical predictors in primigravida. These results show that polygenic risk assessment can inform sCL risk in both primigravida and multigravida and that sCL and gestational duration/sPTB have overlapping genetic contributions.

This study uses a cohort of Black/African American women, who are both underrepresented in genomic studies, and suffer disproportionately higher rates of sCL and sPTB. Conducting this research in a high-risk population offers the highest likelihood that the results could be of clinical utility to patients who match this cohort's demographics. PGSs improve prediction of sCL over clinical risk factors alone, and greatly outperform clinical predictors in primigravida.

In summary, longitudinal models described a constant cervical length until week 25 of gestation which followed a subsequent nonlinear decline up to delivery. sPTB status was associated with a more rapid decrease in cervical length which began earlier in pregnancy. Using Genome-wide Complex Trait Analysis (GCTA) methods, these changes in cervical length were found to be substantially influenced by genetic factors (heritability=51%). Furthermore, the genetic factors influencing cervical change overlapped with genetic influences for pregnancy duration, demonstrating a shared genetic architecture. The strongest genetic correlation was seen with the overall linear change in cervical length (p=0.98) and less so for the genetic correlation with mid-trimester values (p=0.71) and aspects of non-linear change that are most pronounced at the end of pregnancy (p=−0.49). SNP-level associations were observed near genes in progesterone, estrogen, and insulin signaling pathways.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A method of identifying and treating a woman for potential risk of preterm birth associated with mid-trimester clinical short cervix, comprising:
detecting in a biological sample from the woman prior to the women being in a mid-trimester of pregnancy a plurality of single nucleotide polymorphisms;
calculating a polygenic risk score (PGRS) for the woman based on the detected plurality of single nucleotide polymorphisms; and
treating, based on the calculated PGRS, the woman prior to the mid-trimester of pregnancy for the woman with one or more of a surgical procedure of cervical cerclage, or vaginally delivered progesterone.

2. The method of claim 1, wherein additional risk features include one or more of ancestry, ethnicity, age, weight, height, body mass index (BMI), previous live births, previous spontaneous preterm births (sPTB), abortions, miscarriages and a diagnosis of diabetes

3. The method of claim 1, wherein the woman is of African ancestry.

4. The method of claim 1 wherein the woman is primigravida.

5. The method of claim 1 wherein the woman is not pregnant during the detecting and calculating steps.

6. The method of claim 1 wherein the detecting is performed using whole genome sequencing.

7. The method of claim 1 wherein the detecting is performed using a microarray specific for said plurality of single nucleotide polymorphisms.

8. The method of claim 1 wherein the detecting is performed using polymerase chain reaction amplification of a suite of genes or genetic markers.

9. The method of claim 1 wherein calculating is performed using polymorphism specific weights for different single nucleotide polymorphisms.

10. The method of claim 1 wherein the biological sample is blood, plasma or buffy coat.

11. The method of claim 1 wherein the biological sample is saliva.

12. The method of claim 1 wherein the biological sample is a cervical or vaginal sample.

13. The method of claim 1 further comprising detecting a level of Saccharibacteria TM7-H1 in the vaginal sample and comparing of the detected level of Saccharibacteria TM7-H1 to a standard control, and wherein treating is based on both the calculated PGRS and the comparison of the detected Saccharibacteria TM7-H1 to the standard control.

14. The method of claim 1 further comprising detecting a level of Saccharibacteria TM7-H1 in a vaginal sample obtained from the woman, and
comparing of the detected level of Saccharibacteria TM7-H1 to a standard control, and wherein treating is based on both the calculated PGRS and the comparison of the detected Saccharibacteria TM7-H1 to the standard control.

15. The method of claim 1 further comprising a fetal fibronectin assay.

16. The method of claim 1, wherein the calculating step comprises
a) extracting, into a diagnostic model, genetic features from a biological sample of the subject;
b) mathematically integrating the genetic features into the diagnostic model to output the PGRS.

17. A method of determining a risk of mid-trimester clinical short cervix (sCL) for a woman in need thereof and treating the woman, comprising
(a) identifying the woman as being at risk for preterm birth by:
(1) detecting in a biological sample from the subject the presence or absence of alleles of common allelic variants associated with preterm birth, wherein the step of detecting is performed during a first trimester of pregnancy;
(2) calculating a polygenic risk score (PGRS) for said subject based on the presence or absence of the alleles of common allelic variants;
(3) identifying the subject as being at risk for preterm birth when the PGRS score differs from at least one control PGRS score; and
(b) treating the woman identified as being at risk for preterm birth by one or more of administering progesterone, bed rest, cervical cerclage, or cervical pessary.

18. A method for determining a risk of preterm birth for a woman in need thereof and treating the woman, comprising
(a) identifying the subject as being at risk for preterm birth by:
(1) detecting in a biological sample from the subject the presence or absence of alleles of common allelic variants associated with preterm birth, wherein the step of detecting is performed during a first trimester of pregnancy;
(2) calculating a polygenic risk score (PGRS) for said subject based on the presence or absence of the alleles of common allelic variants;
(3) identifying the subject as being at risk for preterm birth when the PGRS score differs from at least one control PGRS score; and
(b) treating the subject identified as being at risk for preterm birth by one or more of administering progesterone, bed rest, cervical cerclage, or cervical pessary.
